(19) United States
(12) Patent Application Publication
(10) Pub. No.: US 2025/0058005 A1
(43) Pub. Date: Feb. 20, 2025

(54) TREATMENT METHOD AND CLEANING APPARATUS FOR IMPLANT TREATED WITH REAGENT, AND METHOD AND APPARATUS FOR CLEANING RESIDUAL REAGENT ON IMPLANT

(71) Applicant: VENUS MEDTECH (HANGZHOU) INC., Hangzhou (CN)

(72) Inventors: Caili MA, Hangzhou (CN); Dajun Kuang, Hangzhou (CN); Lingying Wu, Hangzhou (CN); Yuting Zhu, Hangzhou (CN); Hanqin Gu, Hangzhou (CN); Jesse Jun Qi, Hangzhou (CN); Ken Meng, Hangzhou (CN)

(21) Appl. No.: 18/938,524

(22) Filed: Nov. 6, 2024

Related U.S. Application Data
(63) Continuation of application No. PCT/CN2023/092435, filed on May 6, 2023.

(30) Foreign Application Priority Data
May 6, 2022 (CN) 202210488887.8

(51) Int. Cl. A61L 2/16

(52) U.S. Cl. CPC A61L 2/16

(57) ABSTRACT

A treatment method and a cleaning apparatus for an implant treated with a reagent arc disclosed. The cleaning apparatus for treating an implant treated with a reagent includes: a base; a rotatable rack for placing one or more implants; and a driving mechanism coupled with the rotatable rack.

### CROSS-REFERENCE TO RELATED APPLICATIONS

The present application is a Continuation Application of PCT Application No. PCT/CN2023/092435, filed on May 6, 2023, which claims the priority of Chinese Patent Application No. 202210488887.8, filed on May 6, 2022, the entire contents of which are hereby incorporated by reference.

### TECHNICAL FIELD

The present disclosure relates to the field of medical engineering, and in particular to a treatment method and a cleaning apparatus for an implant treated with a reagent, and a method and apparatus for cleaning a residual reagent on an implant.

### BACKGROUND

Implants may be, for example, prosthetic blood vessels, valve prosthesis, etc., which may be finished products, semi-finished products or components. In order to improve the physical and chemical properties of implants, the implant is usually modified by treatment, during which it may come into contact with chemical reagents and cause reagent residues, which may lead to calcification of the implant, thrombosis and other problems, affecting the biocompatibility of the implant and existing safety risks.

### SUMMARY

The present disclosure provides a treatment method for an implant treated with a reagent, including cleaning the residual reagent on the surface of the implant.

In the following, several alternatives are provided, but merely as further additions or preferences, instead of as further limitations to the above-mentioned technical solution. Without technical or logical contradiction, the alternatives can be combined with the above-mentioned technical solution, individually or in combination.

Optionally, an adhesion force is formed between the residual reagent and the surface of the implant, and the step of cleaning the residual reagent includes applying an external force to overcome the adhesion force.

Optionally, the external force is a friction force.

Optionally, the external force is an adsorption force generated based on the adsorption of a load.

Optionally, the external force is a centrifugal force.

Optionally, the external force is generated based on fluid flow.

Optionally, a portion of the material of the implant has internal pores, and the treatment method further includes cleaning the internal pores.

Optionally, a portion of the implant has an interlayer structure and/or an enclosed space, and the treatment method further includes cleaning the interior of the interlayer structure and/or the enclosed space.

Optionally, the step of cleaning includes driving the implant to move relative to the implant until the residual reagent separates from the implant, thereby obtaining the treated implant.

Optionally, the implant is a heart valve prosthesis which includes a stent and leaflets, the stent is a radially deformable tubular structure, an interior of the tubular structure defines a blood flow channel, and the leaflets are connected to the stent to control the degree of opening of the blood flow channel.

Optionally, the driving method is to drive the stent to gain an acceleration, so that the reagent to be cleaned has a tendency to move relative to the implant.

Optionally, the treatment method includes driving the implant to rotate around a rotation axis so that the reagent to be cleaned has the tendency to move relative to the implant.

Optionally, the implant is arranged parallel to or perpendicular to the rotation axis.

Optionally, the implant includes a stent and leaflets, and the implant has opposite inflow and outflow sides according to the control direction of blood flow by the leaflet, in the case of a perpendicular arrangement, the inflow side faces the rotation axis when the implant rotates around the rotation axis.

Optionally, the implant includes a skirt, wherein the skirt faces the rotation axis.

Optionally, the leaflet is supported on a side of the leaflet facing away from the rotation axis.

Optionally, a support that supports the leaflet has a porous structure.

Optionally, the treatment method includes treating a plurality of implants, each of the implants is at the same distance from the rotation axis in the case of a parallel arrangement.

Optionally, the treatment method includes rotating the implant so that the reagent to be cleaned has a tendency to move relative to the implant under the action of the centrifugal force.

Optionally, when rotating the implant, the rotation axis lies outside the implant, without intersecting with the implant.

Optionally, the implant has opposite inflow and outflow sides, and when the centrifugal force is applied, the rotation axis lies on the inflow side of the implant.

Optionally, the implant has an longitudinal axis which is perpendicular to the rotation axis.

Optionally, the rotation axis and the longitudinal axis of the implant are coplanar.

The present disclosure provides a cleaning apparatus for treating an implant treated with a reagent, and the cleaning apparatus includes:
a base;
a rotatable rack for placing one or more implants; and
a driving mechanism coupled with the rotatable rack.

Optionally, the cleaning apparatus further includes a housing fixed to the base, and the rotatable rack is located inside the housing.

Optionally, a side of the housing facing away from the base defines a housing opening, and the housing opening is covered with a cover body.

Optionally, the driving mechanism is installed within the base, and a control element electrically connected to the driving mechanism is installed on the base.

Optionally, the rotatable rack includes:
a support shaft defining the rotation axis;
one or more holder fixed to the support shaft for loading the implant; and
one or more limiting members, each cooperating with the holder and restricting the implant from detaching from the holder along a radial direction of the support shaft.

Optionally, one end of the support shaft is rotatably mounted on the base, and the other end of the support shaft is rotatably inserted into the cover body.

Optionally, the holder is cup-shaped and includes a cup bottom and a side wall surrounding a periphery of the cup bottom, a side opposite to the cup bottom defines a cup mouth, and the limiting member is engaged at the cup mouth.

Optionally, the cup bottom is detachably connected to the support shaft.

Optionally, the cleaning apparatus further includes a connecting member, wherein

the support shaft defines a mounting hole radially penetrating there through, the connecting member passes through the mounting hole; wherein the holders are arranged in pairs, and a pair of holders both connecting to the connecting member.

Optionally, the cup bottom defines holes for passage of the fluid, and the holes are evenly arranged in a circumferential direction at the cup bottom.

Optionally, the side wall is a hollow structure.

Optionally, the limiting member is a cup lid snapping fitted at the cup mouth, to prevent the implant from moving radially outwardly.

Optionally, at least a portion of an outer peripheral wall of the cup lid is provided with an anti-slip member.

Optionally, the cup lid is annular with an opening defined in a central portion, and a positioning structure engaged with the implant is provided on a side of the cup lid opposite to the cup bottom.

Optionally, one of the side wall and the cup lid is provided with a positioning groove, and the other of the side wall and the cup lid is provided with a positioning protrusion engaged with the positioning groove.

Optionally, the positioning groove includes a first groove portion extending parallel to the longitudinal axis of the holder and a second groove portion extending in the circumferential direction of the holder, and the first groove portion communicates with the second groove portion.

Optionally, the positioning structure is an annular groove and/or a plurality of positioning holes.

Optionally, the implant includes a stent and a leaflet, the stent has connecting ears, wherein one of the connecting ears extends out of the corresponding positioning hole.

The present disclosure further provides a treatment method for an implant treated with a reagent, including:
providing a working surface that matches a shape of a site to be cleaned of the implant; and
bringing the working surface into contact with the site to be cleaned, and adsorbing the residual reagent by the working surface.

Optionally, during the adsorption process, the working surface and the site to be cleaned remain relatively stationary.

Optionally, the working surface is moved along a straight line until coming into contact with the site to be cleaned, and during the adsorption process, the working face is only allowed to move relative to the site to be cleaned along the straight line.

Optionally, during the adsorption process, the working surface makes a relative movement with the site to be cleaned in the circumferential direction of the implant.

Optionally, the working surfaces are arranged in pairs, and a pair of working surfaces are located on both sides of the site to be cleaned.

Optionally, the site to be cleaned is in a shape of a membrane, and a pair of working surfaces are located on both sides of the site to be cleaned in a thickness direction.

Optionally, the treatment method for the implant treated with the reagent is implemented in a working space where an internal air pressure is lower than the external air pressure.

The present disclosure provides an implant cleaning apparatus, including a mold, wherein the mold has a working surface that matches the shape of the site to be cleaned of the implant, and the working surface is made of adsorbent material and/or covered with adsorbent material.

Optionally, the mold includes first and second molds that cooperate with each other, and the working surfaces of the first and second molds are opposite and located on two opposite sides of the site to be cleaned of the implant.

Optionally, the mold further includes a pedestal, when in use, one of the first and second molds is the upper mold and the other is the lower mold, and the pedestal is fixed to a bottom of the lower mold.

Optionally, an outer periphery of the lower mold has a positioning step for supporting one axial end of the implant.

Optionally, the mold further includes a container, wherein the lower mold is placed in the container, and a top of the container is open for the upper mold to enter and exit.

Optionally, both the first and second molds are at least partly cylindrical with the axial end defining the corresponding working surface.

Optionally, the working surface matches the shape of the leaflet of the implant.

Optionally, the inflow side of the leaflet is form-fitted to the lower mold, and the outflow side is form-fitted to the upper mold.

Optionally, the working surface of the lower mold includes at least two slope surfaces arranged in a circumferential direction, a ridge is formed between adjacent slope surfaces, and all the slope surfaces intersect at a central portion of the working surface.

The present disclosure provides a method for cleaning a residual reagent on an implant, the method including driving a fluid to act on the implant for cleaning; wherein the fluid is driven by at least one of positive pressure and negative pressure.

Optionally, before driving fluid to act on the implant, the implant is pre-treated by wiping and/or shaking cleaning.

Optionally, the fluid acts on the implant for 5 seconds to 9 minutes.

Optionally, the method further includes performing a drying process after the cleaning is completed, wherein the drying process includes applying a dry airflow to the implant.

Optionally, the cleaning and/or the drying process is performed in an isolation space.

Optionally, the implant is kept in a liquid environment during the cleaning process.

The fluid is a gas phase fluid and/or a liquid phase fluid;
when driven by positive pressure, the pressure is 0 to 101 KPa, for example, 40 KPa to 70 KPa; and
when driven by negative pressure, the pressure is 0 to −101 KPa, for example,-20 KPa to −70 KPa.

Optionally, the fluid is driven by positive pressure, and the fluid is pre-filtered, for example, by using a filter membrane with a pore size of 0.22-0.45 μm.

Optionally, the fluid is driven by positive pressure, and the method further includes recovering the fluid by gravity recovery and/or negative pressure recovery.

Optionally, the implant has two opposite sides, with one side being a front side facing the fluid and the other side being a back side; and during the cleaning process, a supporting force is applied to the back side of the implant to stabilize the same.

Optionally, during the cleaning process, at least one of the following is changed:
one being the direction of the fluid; and
the other being the spatial posture of the implant.

Optionally, the fluid flows in a concentrated or divergent manner.

Optionally, the fluid is one or more of the following: physiological saline, PBS buffer, and purified water.

Optionally, the fluid includes a first fluid driven by positive pressure and a second fluid driven by negative pressure, wherein the first fluid and the second fluid act on the implant simultaneously or alternately.

Optionally, during the cleaning process, the pressure of the fluid is constant or has a trend of change.

Optionally, the implant is an assembled finished product or intermediate, and the intermediate may be an independent component or a semi-finished product of the assembly or processing.

Optionally, the implant is a valve prosthesis or a valve prosthesis intermediate, such as a heart valve prosthesis. The valve prosthesis intermediate includes but is not limited to a stent, a leaflet, a skirt, an auxiliary component for preventing peripheral leakage or for positioning, etc. The auxiliary component for positioning is used to interact with peripheral tissues in the body to position the valve prosthesis.

Optionally, the valve prosthesis is a dried valve prosthesis, which includes a stent of a meshed cylindrical structure and a seal member connected to the stent. The seal member is a leaflet and/or a skirt, and the stent is a single-layer structure in the radial direction.

When cleaning, negative pressure or positive pressure is used to drive the gas phase fluid to flow.

Optionally, the valve prosthesis is a wet valve prosthesis, or a dry valve prosthesis before drying, the valve prosthesis includes a stent of a meshed cylindrical structure and a seal member connected to the stent, the seal member is a leaflet and/or a skirt, and the stent is a single-layer structure in the radial direction;

When cleaning, positive pressure is used to drive the gas phase fluid and/or liquid fluid to flow.

Optionally, the valve prosthesis is kept in a liquid environment during cleaning.

Optionally, the valve prosthesis includes a stent of a meshed cylindrical structure and a seal member connected to the stent, the seal member is a leaflet and/or a skirt, and at least a portion of the seal member is made of fabric and/or porous material; when cleaning, only gas phase fluid is used; or gas phase fluid combined with liquid phase driven by fluid positive pressure is used, wherein the gas phase fluid is driven by negative pressure and/or positive pressure.

Optionally, the valve prosthesis includes a stent of a meshed cylindrical structure and a seal member connected to the stent, the seal member is a leaflet and/or a skirt, the stent is a multi-layer structure in the radial direction, and the gas phase fluid is driven by negative pressure when cleaning the gap between adjacent layers.

Optionally, cleaning is performed until the amount of the residual glycerol on the implant is less than 450 mg. The present disclosure further provides an apparatus for cleaning a residual reagent on an implant. The apparatus includes a fluid driving device, a delivery pipeline and a working head communicated in sequence; the working head has fluid passage ports, and the fluid driving device drives the fluid to be output or inhaled through the fluid passage ports.

Optionally, the material of the working head is selected from one or more of the following: silicone, rubber, sponge, TPU, and PU.

Optionally, a control valve is provided in the delivery pipeline adjacent to the working head, for example, the control valve is used to adjust the flow rate of the fluid.

Optionally, the working head is divided into the following sections which are docked and communicated with each other from a proximal end to a distal end:
a connecting section, detachably connected to the delivery pipeline; and
an extension section, provided with the fluid passage ports.

Optionally, the fluid passage ports are:
located at the periphery of the extension section; and/or
located at the distal end surface of the extension section.

Optionally, the extension section is a nested double-barrel structure, with a fluid channel defined between the double barrels, and the fluid passage ports are:
located in the inner cylinder wall of the double-barrel structure; and/or
located in the outer cylinder wall of the double-barrel structure; and/or
located in the distal end surface of the double-barrel structure.

Optionally, the distal end of the extension section is made of a porous material, and the pores of the porous material serve as the fluid passage ports. The porous material (block or strip) has a pore size of 100 μm to 1000 μm.

Optionally, the extension section is an enlarged head relative to the connecting section, and the distal end of the enlarged head is flat or is a curved surface structure.

Optionally, the extension section is a tubular structure, and the tubular structure is straight or gradually tapered at the distal end.

Optionally, the apparatus further includes an implant positioning tool.

Optionally, the implant positioning tool includes:
a support table; and
a positioning structure installed on the support table and engaged with the implant.

Optionally, the implant includes a stent of a meshed cylindrical structure and a leaflet connected to the stent, and the positioning structure includes at least one of the following: slots, hooks, and clamps.

The positioning structure acts on at least one of the frame bars, sharp corners, connecting ears, and mesh gaps of the stent.

Optionally, a fluid recovery hole is defined on the support table.

Optionally, the delivery pipeline is configured with a control valve.

Optionally, the number of working heads is one or more, each of the working head is independently configured with the delivery pipeline, or the delivery pipeline includes a main pipe connected to the fluid driving device and branch pipes connected in parallel to the main pipe, with each working head communicating with the corresponding branch pipe.

Optionally, the delivery pipeline is configured with a buffer container.

Optionally, one section of the delivery pipeline is a hose, the length of the hose is 10 cm-20 m, and the hose is adjacent to the working head.

Optionally, the fluid driving device is a positive pressure pump, and the delivery pipeline is further configured with a filter.

Optionally, the apparatus further includes a detection device for detecting at least one of the following: flow rate, flow volume, pressure, and temperature of the fluid.

Optionally, the device further includes a fluid recovery device, wherein the fluid recovery device includes:
a confluence device with an inlet and an outlet, wherein the inlet is used to recover the fluid; and
a recovery container with a liquid collection port and a liquid discharge port, wherein the liquid collection port communicates with the outlet.

Optionally, the confluence device is funnel-shaped, with a protective cover provided at the inlet of the confluence device.

Optionally, the positive pressure pump has a fluid input port and a fluid output port, wherein the fluid output port communicates with the delivery pipeline, and the apparatus further includes a cleaning fluid supply device that communicates with the fluid input port. Optionally, the fluid driving device is a negative pressure pump having a fluid input port for generating negative pressure, wherein the fluid input port communicates with the delivery pipeline.

Optionally, the delivery pipeline is further equipped with a gas-liquid separation device, and the capacity of the gas-liquid separation device is 10 mL to 200 L.

Optionally, the fluid driving device is a bidirectional pump with a fluid input port for generating negative pressure and a fluid output port for generating positive pressure, and the working heads are configured in pairs, with one of the paired working heads communicating with the fluid input port and the other communicating with the fluid output port.

The present disclosure further provides a treatment method for an implant treated with a reagent, including:
providing a fluid channel having opposing low pressure and high pressure sides;
fixing the implant within the fluid channel; and
driving the fluid to pass through the fluid channel and act on the site to be cleaned of the implant, such that the residual reagent has a tendency to move relative to the implant under the action of the fluid.

Optionally, the implant has opposite inflow and outflow sides, wherein the outflow side faces the high pressure side and the inflow side faces the low pressure side.

Optionally, the treatment method for the implant treated with a reagent further includes providing support to the implant on the inflow side, wherein a support which provides support allows fluid to pass through by openings or its material structure.

Optionally, the fluid channel is arranged along the direction of gravity, with the high pressure side facing upwardly.

The present disclosure further provides an implant cleaning apparatus, including:
a deflector hood having two opposite sides, one side defining an opening and the other communicating with a fluid pipeline; and
a fixing seat installed inside the deflector hood for carrying an implant. A fluid channel which communicates with the opening and the fluid pipeline is defined on the fixing seat.

Optionally, the deflector hood includes:
a hood bottom, communicating with the fluid pipeline; and
a peripheral wall, distributed around the hood bottom.

Optionally, the fluid channel includes a main channel extending axially and branch channels radiating radially.

Optionally, the hood bottom and the peripheral wall are formed in separate pieces, and the fixing seat includes:
a main body located within the deflector hood; and
a joint connected to the main body and sealingly penetrates the hood bottom, and a protruding portion communicates with the fluid pipeline.

Optionally, a sealing ring for maintaining sealing is provided at the site where the fixing seat contacts the hood bottom.

Optionally, the main body has a supporting portion, and an end surface of the supporting portion abuts against a site to be cleaned of the implant.

Optionally, the outer periphery of the support portion is provided with a positioning step for an end surface of the implant to abut against.

Optionally, a buffer cushion is installed on the positioning step.

Optionally, the end surface of the support portion and the outer periphery of the main body are both provided with first fluid holes, the end surface of the joint are provided with second fluid holes, and each of the first fluid holes communicates with the second fluid hole via the fluid channel.

Optionally, the second fluid hole penetrates the main body along the axial direction and is exposed on one side of the main body facing the support portion to form a third fluid hole.

Optionally, the support portion has a root portion, and the outer periphery of the root portion defines grooves that open radially outwardly, and each groove correspondingly communicates with a third fluid hole.

Optionally, the fluid channel includes a main flow channel that extends axially, and the third fluid hole includes a central hole communicating with the main flow channel and peripheral holes arranged around the central hole.

Optionally, the main body has an annular side wall surrounding the outer periphery of the root portion, with a radial gap defined between the annular side wall and the outer periphery of the root portion, and the peripheral holes of the third fluid hole are located within the radial gap.

Optionally, fourth fluid holes communicating with the fluid channel are defined on the outer periphery of the root portion, and the buffer cushion avoids the fourth fluid holes.

Optionally, fourth fluid holes communicating with the fluid channel are defined on the periphery of the root portion, and the buffer cushion is made of a porous material that allows fluid to pass through.

Optionally, the fluid channel includes branch channels radiating radially, and the branch channels include two groups, with one group communicating with each of the fourth fluid holes and the other group communicating with each of the first fluid holes.

The method and apparatus for cleaning a residual reagent on implants of the present disclosure have at least the following technical effects.

In the disclosure, the adhesion force between the implant and the residual reagent is overcome by applying an external force to the implant, achieving the effect of cleaning the residual reagent on the surface of the implant, thereby reducing the risk of calcification of the implant, reducing the immunogenicity of the implant, and improving the service life of the implant. In addition, there's no need to introduce any extra reagents, making it easy to operate without affecting the performance of the implant. In addition, the cleaning apparatus can clean multiple implants simultaneously, with high efficiency and more uniform cleaning effect.

### BRIEF DESCRIPTION OF DRAWINGS

FIG. 1a is a schematic structural diagram of a treatment method for an implant treated with a reagent according to an embodiment of the present disclosure;

FIG. 1b is a schematic structural diagram of a cleaning apparatus according to an embodiment of the present disclosure;

FIG. 1c is a schematic structural diagram of a treatment method for an implant treated with a reagent according to an embodiment of the present disclosure;

FIG. 1d is a schematic structural diagram of a cleaning apparatus according to an embodiment of the present disclosure;

FIG. 1e is a schematic structural diagram of a cleaning apparatus according to an embodiment of the present disclosure;

FIG. 1f is a schematic structural diagram of a cleaning apparatus according to an embodiment of the present disclosure;

FIG. 1g is a schematic structural diagram of a cleaning apparatus according to an embodiment of the present disclosure;

FIG. 1h is a schematic structural diagram of a holder of a cleaning apparatus according to an embodiment of the present disclosure;

FIG. 1i is a schematic structural diagram showing the coordination relationship between the support shaft and the holder of the cleaning apparatus according to an embodiment of the present disclosure;

FIG. 1j is a schematic flow chart of a treatment method for an implant treated with a reagent according to an embodiment of the present disclosure;

FIG. 1k is a schematic structural diagram of an implant cleaning apparatus according to an embodiment of the present disclosure;

FIG. 1l is a cross-sectional view taking along a line X-X′ of the implant cleaning apparatus in FIG. 1k;

FIG. 1m is a schematic structural diagram of an implant cleaning apparatus according to an embodiment of the present disclosure;

FIG. 1n is a schematic diagram showing the assembly of the cleaning apparatus in FIG. 1g;

FIG. 1o is a schematic diagram showing the assembly of the cleaning apparatus in FIG. 1g;

FIG. 1p is an enlarged view of part A1 in FIG. 1o;

FIG. 2a is a schematic flow chart of a method for cleaning a residual reagent on an implant according to an embodiment of the present disclosure;

FIG. 2b is a schematic structural diagram of an apparatus for cleaning a residual reagent on an implant according to an embodiment of the present disclosure;

FIG. 2c is a schematic structural diagram of an apparatus for cleaning a residual reagent on an implant according to an embodiment of the present disclosure;

FIG. 3a is a schematic structural diagram of an apparatus for cleaning a residual reagent on an implant according to an embodiment of the present disclosure;

FIG. 3b is a schematic structural diagram of a confluence device and a recovery container according to an embodiment of the present disclosure;

FIG. 4 is a schematic structural diagram of an apparatus for cleaning a residual reagent on an implant according to an embodiment of the present disclosure;

FIG. 5 is a schematic structural diagram of an apparatus for cleaning a residual reagent on an implant according to an embodiment of the present disclosure;

FIG. 6 is a schematic structural diagram of an apparatus for cleaning a residual reagent on an implant according to an embodiment of the present disclosure;

FIG. 7 is a schematic structural diagram of an apparatus for cleaning a residual reagent on an implant according to an embodiment of the present disclosure;

FIG. 8a is a schematic structural diagram of a gas-liquid separation device according to an embodiment of the present disclosure;

FIG. 8b is a schematic diagram of the cleaning environment of a valve prosthesis according to an embodiment of the present disclosure;

FIG. 9 is a schematic structural diagram of a working head according to an embodiment of the present disclosure;

FIG. 10 is a schematic structural diagram of a working head according to an embodiment of the present disclosure;

FIG. 11 is a schematic structural diagram of a working head according to an embodiment of the present disclosure;

FIG. 12 is a schematic structural diagram of a working head according to an embodiment of the present disclosure;

FIG. 13 is a schematic structural diagram of a working head according to an embodiment of the present disclosure;

FIG. 14 is a schematic structural diagram of a working head according to an embodiment of the present disclosure;

FIG. 15 is a schematic diagram of the coordination relationship between the working head and the valve prosthesis according to an embodiment of the present disclosure;

FIG. 16 is a schematic structural diagram of a valve prosthesis according to an embodiment of the present disclosure;

FIG. 17 is a flow chart of a treatment method for an implant treated with a reagent according to an embodiment of the present disclosure;

FIG. 18 is a schematic cross-sectional view of an implant cleaning apparatus according to an embodiment of the present disclosure;

FIG. 19 is an enlarged view of the fixing seat in FIG. 18;

FIG. 20 is a schematic structural diagram of the fixing seat in FIG. 18;

FIG. 21 is a schematic structural diagram of the fixing seat in FIG. 18 viewed from another angle;

FIG. 22 is an exploded view of the fixing seat in FIG. 20;

FIG. 23 is a cross-sectional view of the fixing seat in FIG. 20;

FIG. 24 is a side view of the right-hand assembly portion in FIG. 23.

The reference signs in the figures are described as follows:
100, fluid driving device; 101, fluid input port; 102, fluid output port; 103, negative pressure input port; 111, compressed air pump; 112, liquid pump; 121, negative pressure pump; 130, bidirectional pump;
200, delivery pipeline; 201, main pipe; 202, branch pipe; 203, control valve; 204, control valve; 205, pipeline filter; 220, buffer container; 230, hose; 240, filter;
250, gas-liquid separation device; 251, fluid inlet; 252, fluid outlet; 253, first flow pipe; 254, second flow pipe;
300, working head; 305, fluid passage port; 306, fluid; 307, fluid channel; 3071, inner barrel wall; 3072, outer barrel wall;
310, connecting section; 311, insertion port; 320, extension section; 321, distal end surface; 400, pressure gauge;
500, fluid recovery device; 510, confluence device; 511, inlet; 512, outlet; 513, protective cover; 520, recovery container; 521, liquid collection port; 522, liquid discharge port; 530, liquid tank; 600, cleaning fluid supply device;
700, valve prosthesis; 701, stent; 702, leaflet; 703, inflow side; 704, outflow side; 710, frame bar; 720, sharp corner; 730, connecting ear; 740, mesh gap;
800, cleaning apparatus; 810, base; 811, control element; 820, rotatable rack; 821, support shaft; 822, turntable; 8221, positioning part; 823, limiting member; 824, holder; 825, cup bottom; 8251, hole; 826, side wall; 8261, positioning groove; 8262, first groove portion; 8263, second groove portion; 827, cup lid; 8271, anti-slip member; 8272, positioning protrusion; 828, opening; 829, positioning hole; 830, driving mechanism; 840, housing; 841, housing opening; 842, cover body;
900, cleaning apparatus; 910, first mold; 911, first working surface; 920, second mold; 921, second working surface; 922, slope surface; 923, ridge; 924, central portion; 925, positioning step; 930, pedestal; 940, container;
950, cleaning apparatus; 960, deflector hood; 961, opening; 962, fluid pipeline; 965, hood bottom; 966, peripheral wall; 970, fixing seat; 971, fluid channel; 980, main body; 981, first fluid hole; 982, supporting portion; 983, third fluid hole; 984, positioning step; 985, groove; 986, buffer cushion; 987, radial gap; 988, fourth fluid hole; 990, joint; 992, second fluid hole.

### DESCRIPTION OF EMBODIMENTS

The technical solutions according to the embodiments of the present disclosure will be described clearly and fully in combination with the drawings according to the embodiments of the present disclosure. Obviously, the described embodiments are not all embodiments of the present disclosure, but only part of the embodiments of the present disclosure. Based on the disclosed embodiments, all other embodiments obtained by those skilled in the art without creative work fall into the scope of this invention.

It should be noted that, when a component is “connected” with another component, it may be directly connected to another component or may be indirectly connected to another component through a further component. When a component is “provided” on another component, it may be directly provided on another component or may be provided on another component through a further component.

Unless otherwise defined, all technical and scientific terms used herein have the same meaning as commonly understood by a person skilled in the art. The terms in the description of the present disclosure are used to describe specific embodiments, and not to limit the present disclosure. The term “and/or” used herein includes one or more of the listed options in any combinations, or the combination of all of the listed options.

In this disclosure, the terms “include”, “comprise” and “have”, as well as any variations thereof are intended to cover non-exclusive inclusions, for example, a system, product or device including a series of units is not necessarily limited to those units explicitly listed, but may include other units not explicitly listed or inherent to these products or devices.

Depending on the materials used, the implants to be cleaned in the embodiments of the present disclosure may be mechanical implants, biological implants, polymer implants, or tissue engineering implants, which may be implanted by surgical or interventional methods.

Taking a valve prosthesis as an example, the implantation site may be any one of the following: the aortic valve, the mitral valve, the tricuspid valve, the pulmonary valve, and the venous valve. Before cleaning, depending on the storage method, the valve prosthesis may be in a wet or dry state. The valve prosthesis intermediate may include, for example, a leaflet, a skirt, and a stent. The material of the leaflet or skirts may be any one of biological tissue materials, polymer materials, or tissue engineering materials.

An embodiment of the present disclosure provides a treatment method for an implant treated with a reagent, including cleaning the residual reagent on the surface of the implant.

The embodiments of the present disclosure do not limit the specific types of the reagents, which include chemical reagents that may come into contact with the implant and cause residues during the modification or post-processing of the implant. The reagent treatment is not strictly limited to the specific means or processes of the treatment, such as glutaraldehyde cross-linking fixation, or drying treatment, etc. The reagent may have a certain viscosity, so there will be residue after treatment. If this happens, there will be certain safety risks when the implant enters the human body. As such, it is necessary to remove these residual reagents.

In the prior art, the cleaning of a residual reagent, for example, residual glycerol introduced in the preparation of the dry valve prosthesis, may be neglected. However, this may bring about corresponding biological toxicity if the amount of the residual reagent is too high.

The material of the implant may include metal, biomaterials or polymer synthetic materials, among which biomaterials and some polymer synthetic materials have internal pores, such as the gaps between the interweaving threads in the case that the implant is made of fabric materials, or the internal space of micropores in the case that the implant is made of porous materials.

The internal pores are hard to clean by simply wiping compared with the surface of the implant, and a driving force that acts on the internal pores is required to remove the residual reagent. Based on this, the treatment method of some embodiments of the present disclosure further includes cleaning the internal pores. Some specific methods are provided below.

In addition, some implants have a portion with an interlayer structure and/or an enclosed space. For example, the interlayer structure is composed of multiple layers of stacked sheets, and the stacking may result in more residual reagents between adjacent sheets. The enclosed space is generally formed by the enclosure of sheets, in which fluids can permeate to remove the residual reagents, as such, there may also be a large amount of residual reagents. Based on this, the treatment method of some embodiments of the present disclosure further includes cleaning the interior of the interlayer structure and/or the enclosed space.

The cleaning referred to in this disclosure may include adopting various methods to reduce, dilute, partially remove or completely remove the residual reagents on the implant. For example, a residual reagent on the surface of the implant may be removed by wiping, so there are no visible drops of reagents on the surface of the implant. For example, a residual reagent on the surface of the implant may also be removed by fluid driving, centrifugation, etc. as described below.

When cleaning the residual reagent on the implant (including those on the surface and/or internal pores), the residual reagent on the surface of the implant is driven to move relative to the implant until the residual reagent separates from the surface of the implant, to obtain a treated implant.

The method to drive the relative movement between the residual reagent and the implant may be, for example, driving the residual reagent to move relative to and separate from the implant, such as by adsorption, wiping, or through the action of fluid, etc. It may also be driving the implant to move relative to the reagent so that the residual reagent separates from the implant under the action of inertia.

In some embodiments below, the implant, exemplified by a heart valve prosthesis, may be applied to the aortic valve or various atrioventricular valves, etc., and specifically includes a stent 701 and leaflets 702. The stent 701 is a radially deformable tubular structure, the interior of the tubular structure defines a blood flow channel, and the leaflet 702 is connected to the stent 701 to control the opening degree of the blood flow channel. The method of driving the residual reagent to move relative to the implant may specifically be driving the stent 701 to gain an acceleration so that the reagent to be cleaned has a tendency to move relative to the implant.

Due to the characteristics of the reagent, an adhesion force usually formed between the reagent and the surface of the implant. In this embodiment, cleaning the residual reagents on the implant includes applying an external force to overcome the adhesion force. Locally, for the residual reagents distributed in different sites of the implant, the external force needs to overcome the adhesion force, thus, the direction of the external force is different from that of the adhesion force. For example, the direction of the external force is opposite to that of the adhesion force, and the external force is greater than the adhesion force. Overall, after the external force overcomes the adhesion force, the residual reagent is removed from the implant and the cleaning is completed. The applied force may be a friction force, an adsorption force generated based on load adsorption, a centrifugal force, or may be a force generated based on fluid flow.

The load may generate an adsorption force based on physical adsorption and chemical adsorption, for example, the load is a sponge. When the external force applied is a centrifugal force, the inertial force generated by high-speed rotation may be used to overcome the adhesion force. Suction or blowing force may be generated based on the fluid flow, which can form a shear force against the residual reagent. Alternatively, the applied force may be an electrostatic repulsion force brought by the fluid, which overcomes the adhesion force through suction or blowing.

An embodiment of the present disclosure provides a treatment method for an implant treated with a reagent, including the step of driving the implant to rotate around a rotation axis, so that the reagent to be cleaned has a tendency to move relative to the implant.

In this embodiment, a centrifugal force is applied to gradually separate the reagent to be cleaned from the implant, and fluid action may also be combined during the centrifugation process. The implant, which includes a stent 701 and leaflets 702, has a relative inflow side and an outflow side according to the control direction of blood flow by the leaflets. When the implant rotates around the rotation axis, the inflow side of the implant faces the rotation axis.

Taking the aortic valve as an example of implantation site, when the implant is a self-expanding valve, the stent of the self-expanding valve generally has an outward-expanded anchoring portion on the outflow side, with the leaflets and skirts close to the inflow side, for anchoring considerations. As such, when the self-expanding valve is centrifuged, the valve may be placed so that the skirts may be closer to the rotation axis relative to the center of the self-expanding valve. At this time, the centrifugal force at the leaflets and skirts of the self-expanding valve is relatively small, causing less damage to the leaflets. Of course, for balloon-expandable valves or self-expanding valves with other structures, other methods may also be used to reduce damage to the leaflets.

In order to stabilize the posture of the leaflets, support is provided on the side of the leaflets facing away from the rotation axis, and when supporting, the plurality of leaflets may be kept in a closed state or a semi-open state.

The support that provides support may have a porous structure, which facilitates the leakage and discharge of the fluid (the residual reagents).

Furthermore, the treatment method for the implant treated with a reagent includes rotating the implant so that the reagent to be cleaned has a tendency to move relative to the implant under the action of a centrifugal force.

During centrifugal operation, the implant rotates around the rotation axis A-A′. In one embodiment, the rotation axis A-A′ lies outside the implant when rotating the implant, without intersecting with the implant. This facilitates more flexible utilization of the centrifugal force in conjunction with the suture characteristics and extension direction of the leaflet 702, ensuring the cleaning effect and avoiding damage to the leaflets 702. The implant has opposite inflow side 703 and outflow side 704, and when a centrifugal force is applied to the implant, the rotation axis A-A′ lies at the inflow side 703 of the implant, which is more in line with the direction of the fluid (e.g., the blood flow) that the implant is subjected to after implantation into the body, and further minimizes the damage of the centrifugal force to the implant.

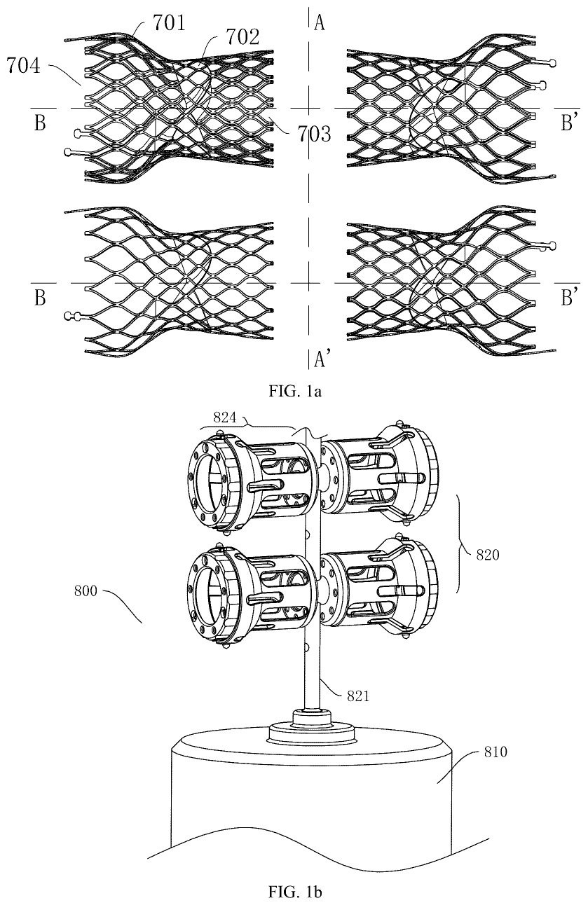

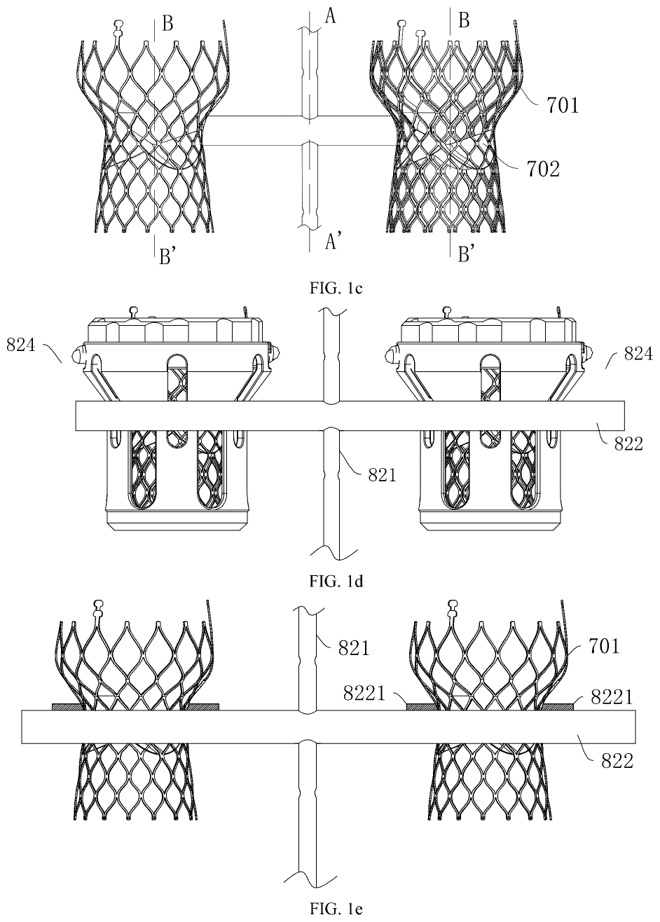

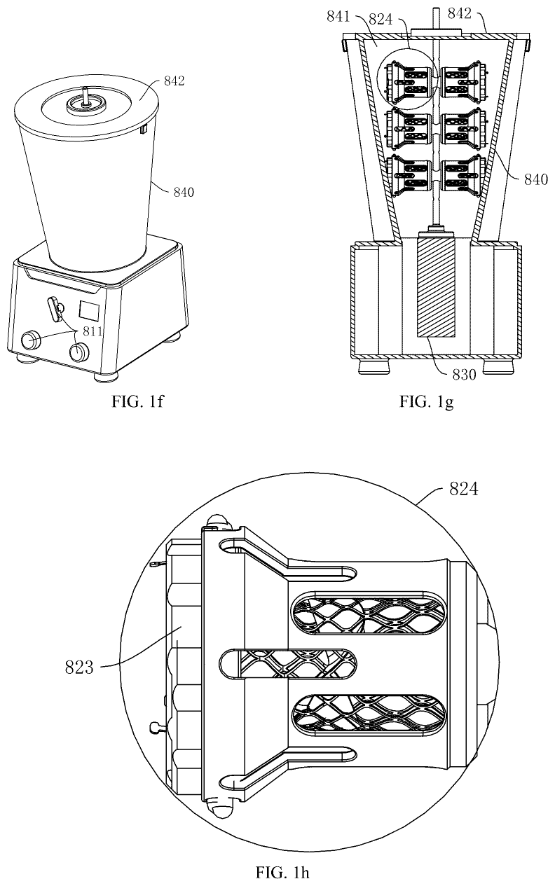

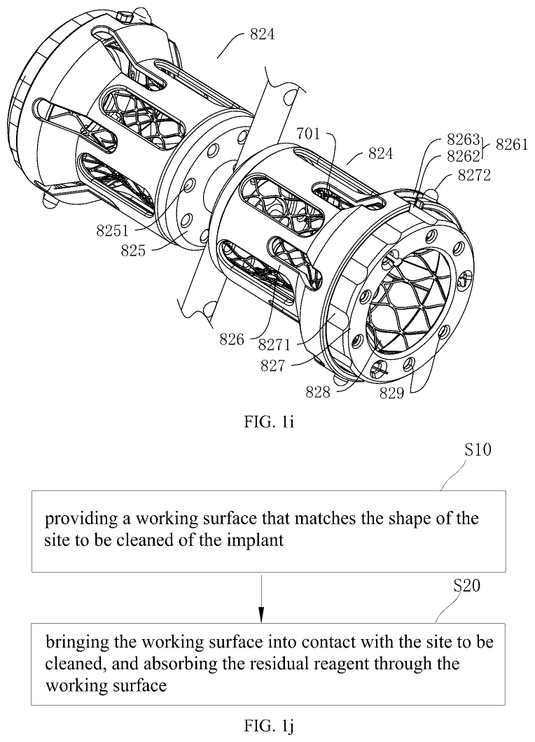

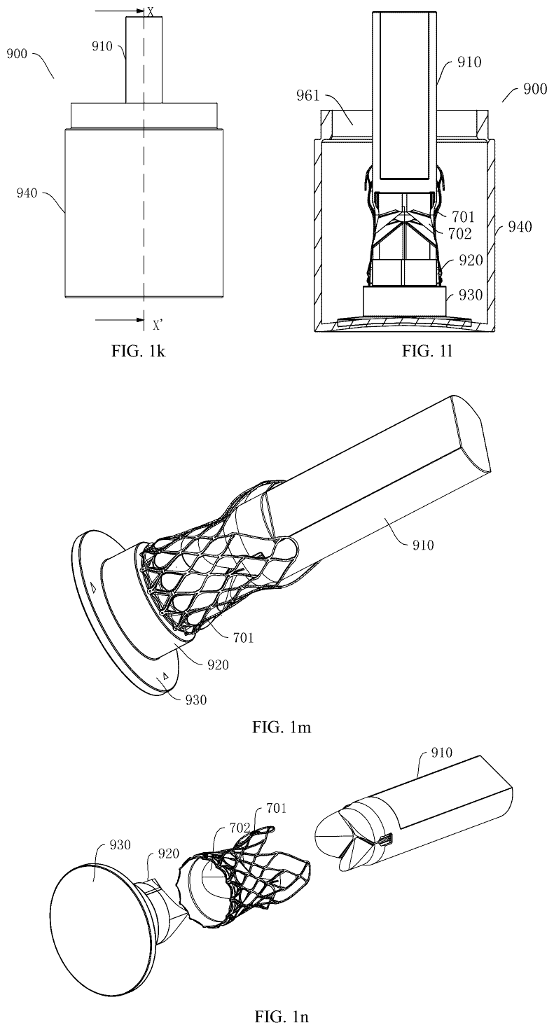

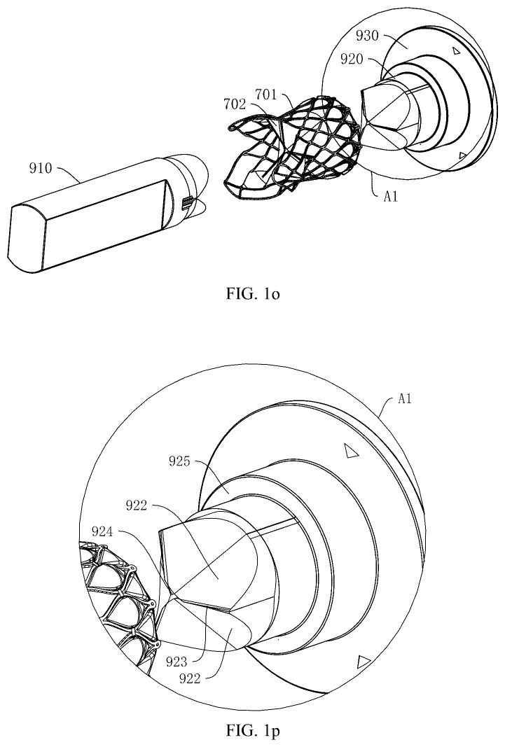

In one embodiment, the implant is arranged perpendicular to the rotation axis. As can be seen in FIG. 1a, the implant has a longitudinal axis B-B′, which is perpendicular to the rotation axis A-A′. Specifically, the rotation axis A-A′ and the longitudinal axis B-B′ are coplanar.

For the vertical arrangement, in the use state, the implant has opposite inflow and outflow sides according to the control direction of the blood flow by the leaflets, the inflow side 703 of the implant is configured for blood inflow, and the outflow side 704 is configured for blood outflow. When the implant rotates around the rotation axis, the inflow side faces the rotation axis. When the implant is driven to rotate, the residual reagent can separate from the implant along the outflow side 704 under the action of centrifugal force.

Furthermore, the leaflet is supported on the side of the leaflet facing away from the rotation axis, so that the leaflet is in a closed state, reducing the risk of tearing. The support for supporting the leaflet has a porous structure. The implant may further include a skirt, which faces the rotation axis.

In order to improve efficiency, a plurality of implants are evenly arranged circumferentially around the rotation axis A-A′ to ensure dynamic balance. Of course, multiple groups of implants may also be arranged along the rotation axis A-A′.

Referring to FIGS. 1c to 1e, in one embodiment, the implant is arranged parallel to the rotation axis. As can be seen in FIG. 1c, the rotation axis A-A′ is parallel to the longitudinal axis B-B′ of the implant. When installed for use, in case the rotation axis is arranged along the direction of gravity, the outflow side may be selected to be below gravity relative to the inflow side, or above gravity as shown in the figure. Furthermore, the treatment method for the implant treated with the reagent includes treating a plurality of implants, and in the case of parallel arrangement, each implant is at the same distance from the rotation axis.

The rotation axis may be defined by the cleaning apparatus, for example, which includes a support shaft 821, and a turntable 822 for fixing the implant. The support shaft 821 defines the rotation axis A-A′. The method of fixing the implant to the turntable 822 may be direct or indirect fixation.

As shown in FIG. 1d, wherein an indirect fixation method is employed, the implant is fixed inside a holder 824, and the holder 824 is fixed to a turntable 822.

As shown in FIG. 1e, wherein a direct fixation method is adopted, the implant (including the stent 701) is held on the turntable 822 by the positioning part 8221.

Referring to FIGS. 1b and 1f through 1h, the present disclosure further provides a cleaning apparatus 800 used for treating an implant treated with a reagent, the cleaning apparatus includes a base 810, a rotatable rack 820 for placing the implant, and a driving mechanism 830 coupled with the rotatable rack 820.

The power source of the driving mechanism 830 may be electric, pneumatic, etc., without strictly limiting the type, and it may be installed, for example, inside the base 810, or may be externally connected to the base 810 through a transmission component or pipeline and may be coupled with the rotatable rack 820. A control device or a transmission mechanism may be configured within the base 810. The rotatable rack 820 has at least one pivot (such as the support shaft 821 below) and is rotatably mounted on the base 810.

The driving mechanism 830 may be, for example, a motor, which is assembled within the base 810 and, when necessary, is in transmission cooperation with the support shaft 821 through a speed regulating mechanism. The transmission mechanism may be, for example, a gear set, and due to the transmission ratio, it also serves as a speed regulating mechanism.

In order to improve protection, the cleaning apparatus 800 further includes a housing 840 fixed to the base 810, and the rotatable rack 820 is located inside the housing 840. The side of the housing 840 facing away from the base 810 defines a housing opening 841 covered with a cover body 842.

When in use, the cover body 842 is fixed to close the housing opening 841. A drainage channel or a collection area may be defined on the housing 840 or the base 810 to facilitate the collection or discharge of the liquid separated by centrifugation. The housing 840, for example, may have a hollow inverted truncated cone structure.

The driving mechanism 830 is installed inside the base 810, and the base 810 is equipped with a control element 811 electrically connected to the driving mechanism 830. For ease of operation, the control element 811 may include a power switch, a speed regulation, a display screen, an I/O interface, etc.

The housing 840 or the cover body 842 may be provided with a liquid injection port, which may be used for flushing while centrifuging. A temperature control device may also be configured inside the housing 840 to ensure a suitable temperature environment. The housing 840 or the cover body 842 may further be provided with a vacuum port for centrifugation under negative pressure or vacuum condition.

The rotatable rack 820 includes: a support shaft 821 which defines the rotation axis A-A′; a holder 824 fixed to the support shaft 821 for loading an implant; and a limiting member 823 cooperating with the holder 824 to restrict the implant from detaching from the holder 824 along the radial direction of the support shaft 821 outwardly.

The support shaft 821 is rotatably mounted on the base 810. In order to facilitate the installation of the holder 824, the top of the support shaft 821 may be further extended, that is, the support shaft 821 has a length sufficient to install a preset number of holders 824. Further, the holders 824 are arranged in multiple groups along the extending direction of the support shaft 821, and each group includes multiple holders 824 evenly arranged in the circumferential direction of the support shaft 821. For example, the multiple holders 824 in each group are symmetrically arranged relative to the support shaft 821 to ensure dynamic balance.

The extended portion of the support shaft 821 is not required to be a round rod, as long as it provides the necessary strength support, there is no strict requirement for its shape.

Referring to FIG. 1i, in the configuration of the cover body 842, one end of the support shaft 821 is rotatably mounted on the base 810, and the other end is rotatably inserted into the cover body 842, to stabilize the configuration and prevent shaking. As such, both ends of the support shaft 821 are respectively limited by the base 810 and the cover body 842. Furthermore, the cover body 842 defines an avoidance hole for the support shaft 821 to be inserted in. A bearing that reduces friction may be installed at the avoidance hole.

The holder 824 is configured to support, mount, or accommodate the implant. In one embodiment, the holder 824 is cup-shaped to accommodate the implant. The holder 824 specifically includes a cup bottom 825 and a side wall 826 surrounding the periphery of the cup bottom 825, with a cup mouth defined on a side opposite the cup bottom 825, wherein the limiting member 823 is detachably connected at the cup mouth. Specifically, the holder 824 is cup-shaped, and its shape matches that of the stent 701, for example, with a flared structure matching the shape of the stent.

The holder 824 is fixed to the support shaft 82, for example, through the cup bottom 825. The cleaning apparatus further includes a connecting member, the support shaft 821 defines a mounting hole radially penetrating there through, and the connecting member passes through each mounting hole. The holder includes a pair of holders both connected to the responding connecting member. The connecting member may be a bolt that is threaded through the cup bottom 825 and secured with a nut.

Specifically, the cup bottom 825 defines an avoidance hole for the connecting member to insert in, and after the connecting member is inserted, the bolt is exposed inside the holder 824. Since the stent of the implant is a tubular structure, this coordinated fixation method will not cause damage to the leaflets of the implant.

In order to facilitate operation and arrangement as needed, the cup bottom 825 is detachably connected to the support shaft 821. As shown in the figure, the holders 824 are installed in pairs by bolts, wherein the side wall 826 is a hollow structure to facilitate weight reduction and fluid dissipation.

Furthermore, the cup bottom 825 defines holes 8251 which facilitate the passage of fluid. The holes 8251, for example, may be evenly arranged at the cup bottom 825 in the circumferential direction.

As for the distribution of the hollow area, the hollow structure includes multiple groups of hollow areas arranged axially and at intervals, with adjacent groups arranged in a staggered manner, and each hollow area is strip-shaped.

The limiting member 823 is a cup lid 827 engaged at the cup mouth, which blocks the implant from moving radially outwardly. The fixing method may use a snap or a thread as shown in the figure for easy disassembly and assembly.

At least a portion of the outer peripheral wall of the cup lid 827 is provided with an anti-slip portion 8271, which may be, for example, a concave-convex structure, anti-slip patterns, etc.

In the case of the snap fixation, one of the side wall 826 and the cup lid 827 defines a positioning groove, and the other of the side wall 826 and the cup lid 827 is provided with a positioning protrusion engaged with the positioning groove. For example, the side wall 826 defines a positioning groove 8261, while the cup lid 827 is fixed with a positioning protrusion 8272.

Furthermore, the positioning groove 8261 includes a first groove portion 8262 extending parallel to the longitudinal axis of the holder 824 and a second groove portion 8263 extending in the circumferential direction of the holder, and the first groove portion 8262 and the second groove portion 8263 are communicated with each other.

When making the snap fixation, the limiting member 823 and the positioning protrusion 8272 are moved along the first groove 8262 toward the cup bottom of the holder 824, till abutting against the second groove 8263. Then, the limiting member 823 and the positioning protrusion 8272 are rotated in the circumferential direction so that the positioning protrusion 8272 enters and is limited in the second groove 8263.

The cup lid 827 is annular, with an opening 828 defined in the central portion to facilitate fluid passage. The cup lid 827 has a positioning structure engaged with the implant on the side opposite to the cup bottom 825. It can prevent displacement of the implant during centrifugation, as well as maintain dynamic balance at the preset installation position. The positioning structure is an annular groove and/or a plurality of positioning holes 829. The end of the stent 701 of the implant has axially protruding corners, such as corners of the frame bar that surround the cells, etc., and each of these corners inserts into a corresponding positioning structure to ensure the positioning effect.

The stent 701 has connecting ears, with one of the connecting ears 730 extending out of the corresponding positioning hole 829. The positioning holes 829 are through holes defined on the cup lid 827, for example, circular holes evenly arranged along the circumference.

The cleaning apparatus of this embodiment can be used to clean multiple implants simultaneously, which greatly improves the cleaning efficiency compared to cleaning a single implant one by one. In addition, the cleaning process is more controllable and precise, which helps to achieve a unified and standardized cleaning effect.

Referring to FIG. 1j, an embodiment of the present disclosure provides a treatment method for an implant treated with a reagent, including:

Step S10, providing a working surface that matches the shape of the site to be cleaned of the implant; and

Step S20, bringing the working surface into contact with the site to be cleaned, and absorbing the residual reagent through the working surface.

During the adsorption process, the working surface and the site to be cleaned remain relatively stationary. The working surface moves along a straight line (such as the longitudinal axis of the implant) to contact the site to be cleaned, and during the adsorption process, the working face is only allowed to move relative to the site to be cleaned along the straight line. On this basis, the working surface may also make relative movement to the site to be cleaned in the circumferential direction of the implant.

The working surface may, for example, be configured to match the shape of the leaflets. An adsorption reagent may, for example, be made of an adsorbent material that covers or cushions the work surface, or be processed from the adsorbent material of the work surface.

Further, the working surfaces are arranged in pairs and are respectively located on both sides of the site to be cleaned. The site to be cleaned is membrane-shaped, and the pair of working surfaces are located on both sides of the thickness direction of the site to be cleaned. The pair of working surfaces may be, for example, the first working surface and the second working surface described below.

The implant includes a stent and leaflets, which have opposite inflow and outflow sides according to the control direction of blood flow by the leaflets. When arranging the pair of work surfaces, one of the paired working surfaces first abuts against the inflow side of the leaflet and then the other one contacts the leaflets.

Furthermore, steps S10 to S20 are implemented in a working space where the internal air pressure is lower than the external air pressure. For example, the working space may be the internal space of a container, where the pressure inside is reduced by suction through a negative pressure pump.

Referring to FIGS. 1k to 1o, the present disclosure provides an implant cleaning apparatus 900, which includes a mold having a working surface that matches the shape of the site to be cleaned of the implant. The working surface is made of adsorbent material and/or covered with adsorbent material. The adsorbent material may cover the surface of the working surface, or cushion between the working surface and the site to be cleaned of the implant.

In order to reduce the risk of injury caused by the contact between the auxiliary tool and the implant during manual operation, as well as to position the implant, this embodiment employs a mold method, which can also improve efficiency.

The adsorbent material is a flexible material, such as a porous material or fabric with good water and oil absorption properties, such as sponge, dust-free cloth, etc.

Generally, the stent 701 has a smooth surface with minimal residual reagents and is thus not a critical site to be cleaned, whereas the leaflets 702 tend to have more residues due to its material characteristics. Given the softness of the leaflets 702, a mold may be used for shape-fitting support.

In order to further improve the working effect, the working surface is provided with a suction hole, a channel connected to the suction hole is defined inside the mold, and the mold is connected to the negative pressure generating device through the channel.

The working surface can provide a larger contact area with the site to be cleaned, allowing for one-time dipping and adsorption. A vacuum pump may also be combined to improve the effect and operating efficiency.

The mold provides support on at least one side of the site to be cleaned, and can also provide support on both sides of the implant to form a clamping effect, while simultaneously adsorbing the residual reagents. For example, in one embodiment, the mold includes a first mold 910 and a second mold 920 that cooperate with each other, and when working, the working surfaces of the first and second molds 910, 920 are opposite and located at opposite sides of the site to be cleaned of the implant.

As shown, the site to be cleaned of the implant is the leaflet 702, the working surface of the first mold 910 is the first working surface 911, and the working surface of the second mold 920 is the second working surface 921. The first and second working surfaces 911, 921 are respectively form-fitted to the opposite sides of the leaflet 702 to be cleaned. The leaflet 702 is a movable part with relatively closed and open states, in this embodiment, the first and second working surfaces 911, 921 are respectively adapted to the corresponding shape of the opposite sides of each of the three leaflets 702 in the closed state.

During operation, the first and second molds 910, 920 may be arranged along the gravity direction and moved relatively to each other. In one embodiment, the implant cleaning apparatus 900 includes a pedestal 930. When in use, one of the first and second molds 910, 920 is a lower mold and the other of the first and second molds 910, 920 is an upper mold. The pedestal 930 is fixed to the bottom of the lower mold. The pedestal 930 and the lower mold may be formed in separate pieces. When the lower mold is stably positioned, the pedestal 930 may also be a part of the lower mold.

Taking the second mold 920 as an example of the lower mold for further explanation, the outer periphery of the lower mold has a positioning step 925, which is used to support one axial end of the implant. Specifically, the positioning step 925 is used to support the stent of the implant.

In order to adapt to implants of different configurations, the distance between the positioning step and the working surface of the lower mold is adjustable. This is achieved, for example, by lifting the working surface or by attaching a washer to the working surface.

The implant cleaning apparatus 900 further includes a lifting mechanism, which is coupled with the upper mold to control the stroke and speed thereof, for example, by an automation device. Furthermore, the lower mold and/or the pedestal are provided with circumferential registration marks for aligning with the upper mold to avoid damage to the leaflets clamped by the two.

The implant cleaning apparatus 900 may further include a container 940 in which the lower mold is placed, with an opening 961 defined in the top of the container 940 for the upper mold to enter and exit. To facilitate processing and holding, in particular to match the internal shape of the stent 701, both the first and second molds 910, 920 are at least partially cylindrical, with each axial end defining corresponding working surface.

The stent 701 of the implant is a tubular structure, the working surfaces of the first and second molds 910, 920 are adapted to fit the leaflets 702 of the implant, and both the first and second molds 910, 920 are partially extended into the tubular structure, that is, at least the extended portions of the first and second molds 910, 920 are cylindrical shapes adapted to the tubular structured stent 701. The ends of both molds extending into the tubular structure and adjacent to the leaflets 702 define the corresponding working surfaces.

Referring to FIG. 1p, in which the second mold 920 is shown as an example of the lower mold for further explanation. The inflow side 703 of the leaflet 702 is form-fitted to the lower mold, and the outflow side 704 is form-fitted to the upper mold. The working surface of the lower mold includes at least two slope surfaces 922 arranged along the circumferential direction, with a ridge 923 formed between adjacent slope surfaces 922, and all slope surfaces 922 intersect at the central portion 924 of the working surface, i.e., the intersection center of each leaflet 702.

Typically, the reagent cleaning method for wet implants is based on shaking cleaning, while the reagent cleaning method for dry implants is based on wiping. Both methods have limitations and are ineffective in reducing residual reagents introduced during the implant processing.

The large amount of residual aldehyde groups may easily cause problems such as implant calcification and thrombosis, and the large amount of residual drying reagents, such as glycerol, affect the biocompatibility of the implant. In case leaflets or skirts are made of porous structural materials such as fabrics, the amount of residual reagents is even greater. Therefore, to ensure that the amount of the residual reagents meets certain acceptable standards, there are certain limitations on the reagent concentration in the treatment solution.

In addition, for implants with a stacked structure, it is difficult to remove the residual reagents between the interlayers by using conventional methods such as wiping, regardless of whether the interlayers form an enclosed or open space. To solve this problem, the current approach is to increase the preclinical cleaning/hydration time or control the amount of reagents such as glycerol. However, these methods either increase the surgical preparation time or have a certain impact on the service life of the implant, and the effectiveness of reagent cleaning is not ideal.

Conventional wiping and cleaning methods are less effective for removing residual reagents on the valve prosthesis. To ensure that the amount of residual reagent meets certain acceptable standards, there are certain limitations on the reagent concentration in the treatment solution, often at the cost of performance, which may affect the service life of the valve prosthesis and increase the time for surgical preparation.

An embodiment of the present application provides a method for cleaning residual reagents on an implant, including driving a fluid to act on the implant for cleaning, wherein the fluid is driven by at least one of positive pressure and negative pressure.

The embodiments of the present disclosure efficiently remove (blow or suck away) residual reagents on the implant by the method of driving fluid, which is particularly suitable for skirts with a porous structure, such as fabric, and a complex stent with a stacked structure, when the implant is a valve prosthesis or a valve prosthesis intermediate (unless otherwise specified, the valve prosthesis hereinafter may also be a valve prosthesis intermediate). As such, the risk of calcification of the implant can be reduced and the immunogenicity of the implant can be decreased, thereby increasing the service life of the implant.

Meanwhile, the method provided in this embodiment can shorten or eliminate the cleaning or hydration time of the implant before surgery by removing residual reagents, which reduces the surgical preparation time and improves the success rate of surgery. In the specific operation, one or more methods provided in the following embodiments may be used to remove the residual reagent.

In different embodiments, unless particularly otherwise described or there is a technical contradiction, the method of driving fluid may be positive pressure driving, negative pressure driving, or a combination of the two. The fluid may be a gas phase fluid, a liquid phase fluid, or a combination of the two.

When driven by positive pressure, the pressure of the fluid is in a range of 0 to 101 KPa (relative to atmospheric pressure), for example, 40 KPa to 70 KPa.

When driven by positive pressure, the pressure is in a range of 0 to −101 KPa (relative to atmospheric pressure), preferably −40 to −70 KPa, and may be −60 KPa, for example.

Combined driving may be achieved by alternately or simultaneously carrying out the positive pressure driving and negative pressure driving throughout the entire process. For different kinds of implants, the fluid acts on the implant for 5 seconds to 9 minutes.

For example, the fluid includes a first fluid driven by positive pressure and a second fluid driven by negative pressure, and the first fluid and the second fluid act on the implant simultaneously or alternately, wherein the first fluid is a liquid phase gas and the second fluid is a gas phase fluid.

In one embodiment, the pressure of the fluid during the cleaning process is constant or has a trend of change.

In case of having a trend of change, the pressure may be adjusted manually or automatically controlled by a software control program. For example, when cleaning residual reagents on biological tissue, the pressure may be adjusted to a lower level (excessive pressure may pull on the biological tissue and affect its performance). When cleaning residual reagents on porous structures (such as foams and fabrics), it is suitable to increase the pressure to quickly and efficiently remove residual reagents between the pores.

The valve prosthesis generally includes components such as a stent, leaflets, and skirts, and there is no strict limitation on the order of cleaning each component.

When a liquid phase fluid is used, one or more of the following: physiological saline, PBS buffer, and purified water, may be selected as the liquid phase fluid. The liquid phase fluid is sterilized before use.

To avoid potential contamination caused by direct discharge of fluid, or to improve operating conditions, the method may further include recovering the fluid through gravity recovery and/or negative pressure recovery. The recovered fluid is discharged or recycled after treatment. The gas phase fluid may directly be composed of air or other gases that do not affect the performance of the implant.

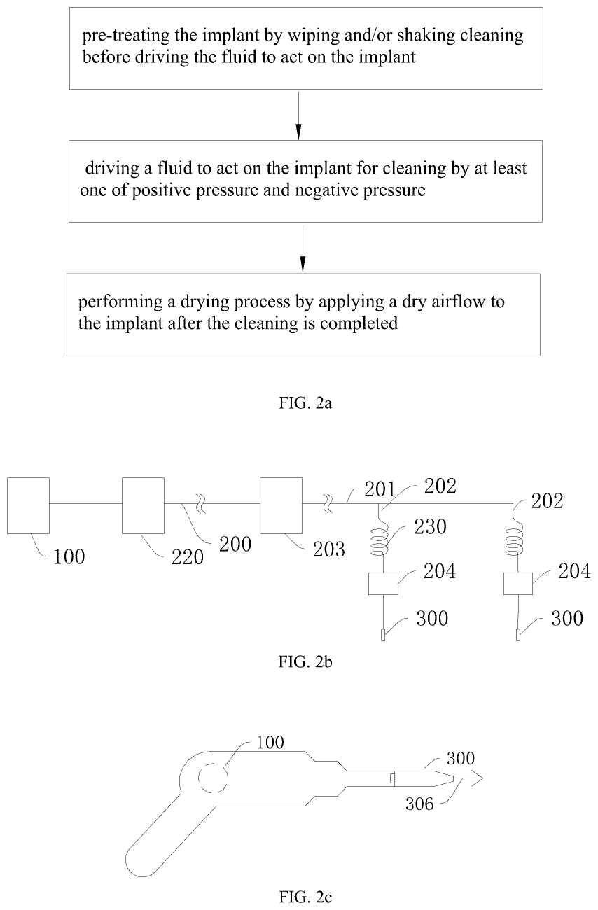

Referring to FIG. 2a, the implant may be pre-treated by wiping and/or shaking cleaning before driving the fluid to act on the implant.

The method may further include a drying process after cleaning is completed (in the case of a valve prosthesis, for example, where the drying process is primarily to remove the residual liquid fluid on the valve prosthesis, rather than to produce a dry valve prosthesis in the conventional sense). The drying process may specifically be carried out by applying a dry airflow to the implant.

The cleaning, pretreatment and drying processes may be carried out in open, semi-open or isolated spaces, respectively.

The residual reagent on the implant may present on the surface only, or may have penetrated into the interior of the material. Depending on the different previous processes, the residual reagent may include glycerol, glutaraldehyde, etc. The standard for completing the cleaning of the residual reagent may be, for example, that the amount of the residual glycerol on the implant is 450 mg or less.

The implant has two opposite sides, one of which is the front side facing the fluid and the other is the back side. To stabilize the position of the implant and ensure uniform cleaning, a supporting force is applied to the back of the implant during the cleaning process.

Regardless of whether positive or negative pressure is applied, there is a possibility of displacement or misalignment of the implant under the action of the fluid. As such, a directional supporting force is applied to the implant downstream of the fluid movement direction.

The supporting force may be provided through auxiliary tooling or even hand-held. The force point of the supporting force is not necessarily required to be on the back of the implant, which should be understood according to the direction of action and the expected effect.

To ensure the cleaning effect, especially for blind spots, the relative position between the fluid and the implant is adjustable during the cleaning process, for example, at least one of the following two is adjustable: one being the direction of the fluid; the other being the spatial posture of the implant. During the cleaning process, one or more fluid sources are configured for the fluid, and the directions of the fluid sources are the same or independently adjustable.

In the case of cleaning by positive pressure liquid, for example, one or more nozzles may be configured with a fixed or adjustable nozzle angle. In addition, for each nozzle, the fluid output flows in a concentrated or divergent manner. Of course, the structure of the nozzle may be configured as required.

When driven by positive pressure, the fluid may be pre-filtered, for example, by a filter membrane with a pore size of 0.22-0.45 μm, to avoid secondary contamination caused by the fluid.

Referring to FIGS. 2b and 2c, in one embodiment, a apparatus for cleaning a residual reagent on an implant is further provided, which including a fluid driving device 100, a delivery pipeline 200 and a working head 300 connected in sequence. The working head 300 has a fluid passage port, and the fluid driving device 100 drives the fluid to be output or inhaled through the fluid passage port. Referring to FIG. 2c, the apparatus for cleaning a residual reagent on an implant provided in each embodiment of the present disclosure may be integrated into a miniaturized handheld device for delivering fluid 306.

In order to facilitate adjustment, the delivery pipeline 200 is configured with a control valve, for example, controlled in a manual or automatic manner. In case of automatic control manner, a signal collection element for signals of flow, pressure or time may be configured accordingly to assist in generating control instructions.

The number of working head 300 is one or more, and each working head 300 is independently configured with the delivery pipeline 200, or the delivery pipeline 200 includes a main pipe 201 connected to the fluid driving device 100 and branch pipes 202 connected in parallel to the main pipe 201, with each working head 300 communicating with the corresponding branch pipe 202. The various parts of the apparatus for cleaning a residual reagent on implants, including the working head 300, may be docked and communicated with each other, for example, through connectors that are easy to disassemble and assemble (such as plug-in connections or threaded connections, using Luer connector, etc.).

Accordingly, the control valve includes a control valve 203 installed on the main pipe 201 and a control valve 204 adjacent to the working head 300. The control valve 204 is installed on each branch pipe 202. The control valve 203 is used to, for example, control the output of the fluid of the overall apparatus, and the control valve 204 is used to adjust relevant parameters such as fluid flow rate and pressure.

In order to maintain pressure stability and reduce fluctuations, the delivery pipeline 200 may be configured with a buffer container 220, which communicates with the delivery pipeline between the fluid driving device 100 and the working head 300. The buffer container 220 may be designed with a capacity according to the processing capacity and equipped with corresponding pressure sensing elements.

In order to facilitate adjusting the posture of the working head, or to adapt to the mobile operation, one section of the delivery pipeline 200 is a hose 230, which is located adjacent to the working head 300 to facilitate flexible operation. The length of the hose 230 is 10 cm to 20 m, and it may be, for example, a spring telescopic hose, a bellows, etc. The material of each part of the delivery pipeline may be selected from one or more of the following: PU, silicone, TPU, PTFE, and stainless steel.

The delivery pipelines are categorized into straight connection pipes and spring connection pipes (understood as length-expanding hoses), for example, by shape. The straight connection pipe is used as the straight path part of the delivery pipeline, while the spring connection pipe is used to connect the various devices in the apparatus to buffer vibration and accommodate assembly errors. When used as laboratory equipment, the length of the straight connection pipe may be 20 cm to 30 cm, and the length of the spring connection pipe may be 30 cm to 1 m, for example 40 cm.

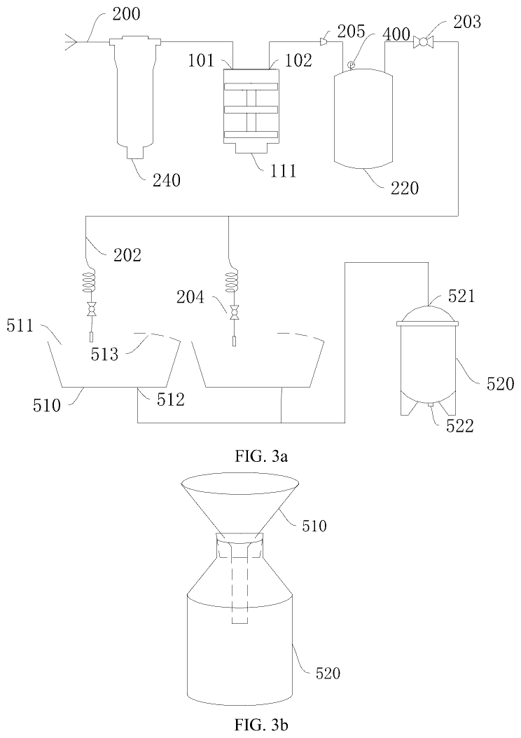

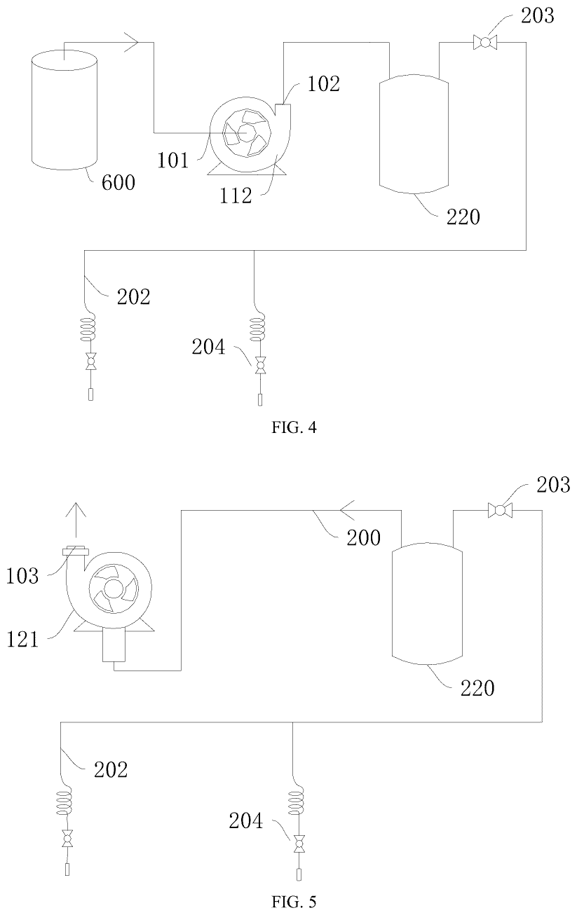

In one embodiment, referring to FIGS. 3a to 4, the fluid driving device 100 is a positive pressure pump, i.e. it drives the fluid by positive pressure. Depending on the type of fluid (gas phase, liquid phase) and flow volume requirements, a corresponding positive pressure pump may be selected. To facilitate driving the positive pressure pump, the apparatus may further include a detection device to detect (and/or display) at least one of the following: flow rate, flow volume, pressure and temperature of the fluid. For example, a sensor or a pressure gauge 400 may be installed on the buffer container 220 to detect the fluid pressure in the delivery pipeline.

Depending on the type of fluid, the positive pressure pump may be a compressed air pump 111 as shown in FIG. 3a, or a liquid pump 112 as shown in FIG. 4. In case of the compressed air pump 111, the delivery pipeline 200 is provided with a filter 240 before connecting to the compressed air pump 111, so as to prevent environmental impurities from flowing into the compressed air pump 111. A pipeline filter 205 may further be installed in the pipeline between the compressed air pump 111 and the buffer container 220. The filter 240 can sterilize and remove impurities from the gas phase fluid, for example, by a filter membrane assembly with a pore size of 0.22 to 0.45 μm, to ensure the cleanliness of the fluid output from the working head 300. In each embodiment of the present disclosure, the arrows marked at the delivery pipeline 200 in the corresponding figure represent the flow direction of the fluid.

Referring to FIGS. 3a and 3b, the apparatus further includes a fluid recovery device 500, which includes a confluence device 510 and a recovery container 520. The confluence device 510 has an inlet 511 and an outlet 512, and the inlet 511 is used for the recovery of fluid 522. The recovery container 520 has a liquid collection port 521 and a liquid discharge port 522, and the liquid collection port communicates with the outlet 512.

For example, the confluence device 510 is funnel-shaped, with a protective cover 513 provided at the inlet 511 of the confluence device. One end of the protective cover 513 is movably arranged at the edge of the inlet 511 of the confluence device 510, shielding at least a part of the inlet 511 and forming a semi-open or isolated space in combination with the confluence device 510.

The protective cover 513 may be made of a transparent material, for example, so as not to block the sight of the operator and to prevent splashing of the reagent removed from the implant. Contrary to FIG. 3a, in which multiple confluence devices 510 communicate with the recovery container 520 through the delivery pipeline for unified processing, the confluence device 510 and the recovery container 520 as shown in FIG. 3b may also be set in a one-to-one form, wherein each working head 300 is equipped with a confluence device 510 and a recovery container 520.

A positive pressure pump, which is configured as the fluid driving device 100, has a fluid input port 101 and a fluid output port 102, wherein the fluid output port 102 communicates with the delivery pipeline 200, and the apparatus further includes a cleaning fluid supply device 600 communicating with the fluid input port 101. In this embodiment, the confluence device 510 and the recovery container 520 are integrally formed or separately arranged. In case of separately arranged, each branch pipe 202 is configured with a confluence device 510, and a liquid collection port 521 of the recovery container 520 communicates with the outlet 512 of each confluence device 510.

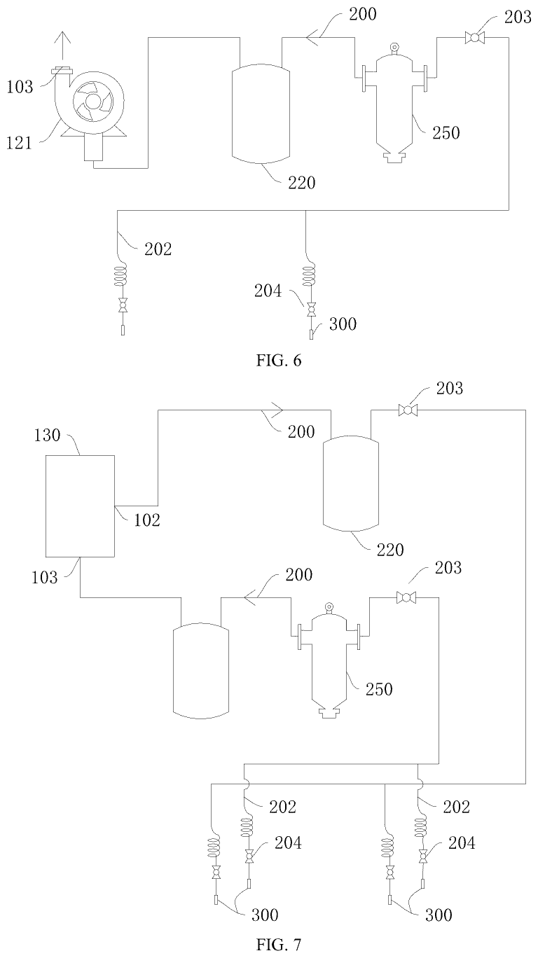

Referring to FIGS. 5 and 6, in one embodiment, the negative pressure pump, which is configured as the fluid driving device 100, has a fluid input port (negative pressure input port 103) that generates negative pressure, wherein the fluid input port communicates with the delivery pipeline 200. The negative pressure pump 121 may be, for example, a vacuum pump. The negative pressure pump can suction the residual reagents on the implant through the negative pressure input port 103.

Referring to FIGS. 6 and 7, in the case that the fluid driving device 100 is equipped with a negative pressure input port 103, a gas-liquid separation device 250 is further configured on the delivery pipeline 200. The gas-liquid separation device 250 separates the inhaled airflow from the liquid phase, which is beneficial for liquid recovery or protecting the device.

The negative pressure input port 103 is located near the implant and can directly suction the residual liquid reagents on the surface of the implant. When there is no visible liquid flow in the gas-liquid separation device 250, it indicates that the cleaning of the site to be cleaned of the implant is completed. Furthermore, the capacity of the gas-liquid separation device 250 may be set according to demand, for example, 10 mL to 200 L.

Referring to FIG. 7, in one embodiment, the fluid driving device 100 is a bidirectional pump 130 which has a fluid input port (negative pressure input port 103) generating negative pressure and a fluid output port 102 generating positive pressure. The working heads 300 are arranged in pairs, with one of the paired working heads 300 communicating with the negative pressure input port 103 and the other communicating with the fluid output port 102. Accordingly, the delivery pipeline 200 includes two parallel pipelines for delivering positive pressure fluid and negative pressure fluid, and each parallel pipeline is provided with a control valve 203 for controlling the main pipe and control valves 204 for controlling the branch pipes 202. The paired working heads 300 may be used in such a way that either one of the two acts on the implant to be cleaned, or both act on opposite sides of the implant simultaneously. For example, the negative pressure input port 103 inputs gas phase fluid, and the fluid output port 102 outputs liquid phase fluid.

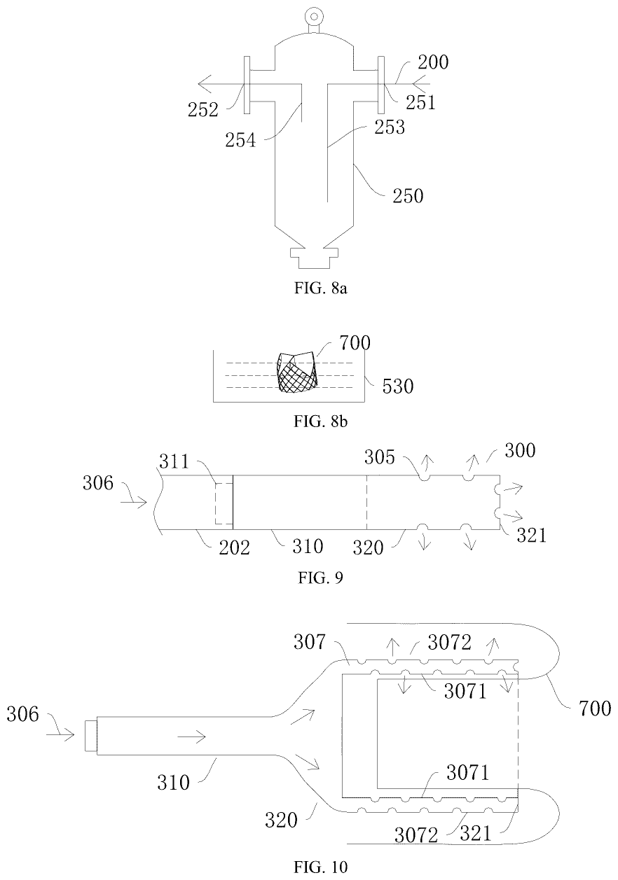

Referring to FIG. 8a, the gas-liquid separation device 250 has a fluid inlet 251 and a fluid outlet 252 that communicate with the delivery pipeline 200. The gas-liquid separation device 250 may be provided with seal plugs at the fluid inlet 251 and the fluid outlet 252, and the seal plugs may be made of materials such as rubber, silicone, etc., to ensure the sealing after connection and avoid fluid leakage. The gas-liquid separation device 250 is provided with a first flow pipe 253 docked and communicated with the fluid inlet 251 and a second flow pipe 254 docked and communicated with the fluid outlet 252. The two flow pipes are separate within the gas-liquid separation device 250, and the first flow pipe 253 extends deeper into the gas-liquid separation device 250 than the second flow pipe 254. The material of each flow pipe may be, for example, one or more of polymer materials such as PP, PE, PTFE, etc.

In one embodiment, referring to FIG. 8b, the implant is cleaned by driving the fluid with positive pressure/negative pressure. During cleaning, the implant is kept in a liquid environment e.g. immersed in a liquid tank 530. The dotted lines in the liquid tank 530 in the figure represent the liquid. The capacity of the liquid in the liquid tank 530 may be, for example, 5 L, and the liquid may be, for example, sterile saline. The implant may be, for example, a valve prosthesis 700. When in use, the working head 300 is placed at the site to be cleaned of the valve prosthesis 700, such as the leaflets, skirts, and other stacked structures of the valve prosthesis.

If driving by positive pressure, the gas phase fluid impacts the surface of the implant to remove the residual reagent in the liquid environment. If driving by negative pressure, the fluid in the fluid tank 530 is sucked into the delivery pipeline through the fluid passage port provided on the working head 300, achieving cleaning of the implant. The residual reagent in various sites of the implant is recovered to the gas-liquid separation device 250 along with the liquid phase fluid. The cleaning time for each site of the implant may be, for example, 30 seconds to 1 minute, and the cleaning time of different sites may be determined according to the remaining amount of residual reagent observed during use.

Regarding the selection of the working head (i.e., the nozzle aforementioned in case of positive pressure driving), the material of the working head 300 is selected from one or more of the followings: silicone, rubber, sponge, TPU, and PU. The shape and structure of the working head may be selected according to factors such as the usage scenario, the type of implant, or the material of each part of the implant. In FIGS. 9 to 14, the arrows in each figure represent the direction of fluid delivery, which is reversible depending on the fluid driving device.

Referring to FIG. 9, the working head 300 may be divided into a connecting section 310 and an extension section 320 from the proximal end to the distal end (the end of the fluid passage port 305 is regarded as the distal end) which are docked and communicated with each other. The connecting section 310 is detachably connected to the delivery pipeline, specifically connected to the branch pipe 202, and the extension section 320 is provided with fluid passage ports 305. According to different application scenarios, the working head 300 may be replaced, disassembled, or alternately used with different structures. The proximal end of the connecting section 310 is provided with an insertion port 311 that is docked and communicated with the branch pipe 202. The extension section 320 of each working head may be made of materials that avoid damage to the implant, such as silicone.

The fluid passage ports 305 are distributed at the periphery of the extension section 320 and/or at the distal end surface 321 of the extension section 320. As shown in FIG. 9, the fluid passage ports 305 are distributed at both the periphery of the extension section 320 and the distal end surface 321.

Referring to FIG. 10, the extension section 320 is a nested double-barrel structure with a fluid channel 307 defined between the double barrels. The fluid passage ports 305 are located: in the inner barrel wall 3071 of the double-barrel structure; and/or in the outer barrel wall 3072 of the double-barrel structure; and/or in the distal end surface 321 of the double-barrel structure. The extension section 320 of the double-barrel structure may be used to clean the valve prosthesis 700 with the interlayer.

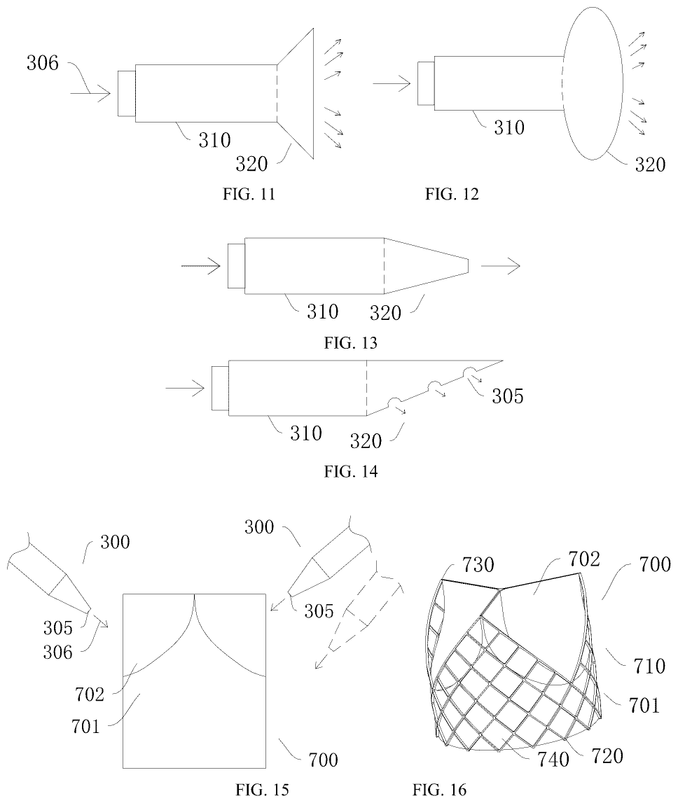

Referring to FIGS. 11 and 12, the extension section 320 is an enlarged head relative to the connecting section 310. The distal end of the enlarged head as shown in FIG. 11 is flush, while the distal end of the extension section 320 as shown in FIG. 12 is a curved surface structure. The enlarged head may be made of a porous material, with the pore thereof serving as the fluid passage port. The porous material may be in the form of blocks, strips, etc., and the pore size thereof is 100 μm to 1000 μm. The porous material may be, for example, a sponge. The porous material can enlarge the contact area between the working head 300 and the implant to be cleaned, allowing for cleaning of the residual reagents on, for example, the porcine pericardial skirts and leaflets of the valve prosthesis. The porous structure is conducive to fluid output and competent for drying the implant after cleaning, which includes wiping and drying the implant or components thereof.

Referring to FIGS. 9, 13 and 14, the extension section 320 is a tubular structure, which is straight as shown in FIG. 9 or gradually tapered at its distal end. The distal end tapering may be in the form of convergence of the peripheral of the distal end toward the axis as shown in FIG. 13, or the form of eccentric convergence of one side of the peripheral of the distal end toward the axis. The tapering distal end of the extension section as shown in FIG. 13 may extend into the interlayer of the stacked implant, to facilitate the removal of the reagent. The extension section as shown in FIG. 14 has a larger distal inclined surface compared to that of FIG. 13, and is suitable for parts with a larger area of the implant, such as the skirts.

Referring to FIG. 15, for the implant to be cleaned, when the fluid 306 acts on the implant, the orientations of the fluid passage ports 305 are the same or each independently adjustable. As shown on the right side of the figure, the fluid passage ports 305 are each independently adjustable, and the orientations thereof are changeable at different stages, for example, from facing the leaflet 702 to facing the stent 701 as shown in the figure.

Referring to FIG. 16, in one embodiment, the apparatus further includes an implant positioning tool. The implant positioning tool includes a support table and a positioning structure. A fluid recovery hole is defined on the support table. The positioning structure is installed on the support table and engages with the implant.

The implant, hereinafter exemplified by a valve prosthesis 700, includes a stent 701 of a meshed cylindrical structure and leaflets 702 connected to the stent. The positioning structure includes at least one of the following: slots, hooks, and clamps. The positioning structure can act on the frame bar 710, the sharp corner 720, the connecting car 730, the mesh gap 740, and other sites of the stent.

According to the implementation scenarios of reagent clearance for valve prosthesis with different structures and states, various embodiments of the present disclosure are auxiliary described as follows.

(1) Clearance of residual reagents on the surface of a dry valve prosthesis with a single-layer structure after drying: When cleaning, the residual reagents on the valve prosthesis surface is removed by the gas phase fluid driven by negative pressure or positive pressure. The valve prosthesis with a single-layer structure may be described as follows: the valve prosthesis 700 includes a stent 701 with a meshed cylindrical structure and a seal member connected to the stent 701, the seal member is a leaflet 702 and/or a skirt, and the stent 701 is a single-layer structure in the radial direction.

(2) Clearance of residual reagents on wet or dry valve prosthesis with a single-layer structure before drying: The gas phase fluid and/or liquid fluid is driven by positive pressure to carry out targeted flushing to the leaflets, skirts, and other stacked structures, the reagents thereon are thus efficiently removed. The valve prosthesis, such as an aortic valve prosthesis made from glutaraldehyde cross-linked porcine pericardium and nickel-titanium alloy, may further be kept in a liquid environment (such as saline) for cleaning. Conventional shaking cleaning and other methods cannot significantly reduce glutaraldehyde residues and bioburden. The pressure of the positive air flow is adjusted to 50 KPa and flows to the working head after passing through a pipeline filter with a pore size of 0.22 μm. The valve prosthesis and the working head are immersed in a liquid tank containing sterile saline. The working head is configured to generate gas phase fluid of the sterile saline to perform targeted flushing to the leaflets, skirts and other stacked structures, and the flushing is performed 3 times for 3 minutes each time. In this way, the residual glutaraldehyde and bioburden can be significantly reduced with amounts far below acceptable standards, which reduces the risk of calcification and thrombosis after implantation, thereby improving the service life and safety of the valve prosthesis.

(3) Clearance of reagents on skirts and/or leaflets with porous structures such as fabrics: In this case, gas phase fluid or gas phase fluid combined with liquid fluid driven by negative pressure/positive pressure is used for flushing. The seal members of valve prosthesis 700 are skirts and/or leaflets 702, and at least a portion of the seal member is made of fabric and/or porous material. When cleaning, only gas phase fluid is used; or gas phase fluid combined with liquid fluid driven by positive pressure is used, wherein the gas phase fluid is driven by negative pressure and/or positive pressure. For example, when treating a valve prosthesis with skirts made of PET fabric, which has a large amount of reagents such as glycerol residual thereon, first, the flowing liquid on the surface of the valve prosthesis is wiped initially. Then, the working head is used to further remove the residual reagent from the surface of the valve prosthesis under a pressure of −50 KPa. The working head may be pressed against each of the site to be cleaned and held for 30s to 1 min. The reagents on the surface of the fabric will automatically flow to the delivery pipeline through the reagent collection tube under the action of negative pressure. Once there is no obvious liquid flow in the delivery pipeline, one can continue to remove the reagents at the next site. The negative pressure cleaning method can achieve the surface of the valve prosthesis made of PET fabric in a relatively dry state (by sucking the surface moisture). Meanwhile, the leaflets of the porcine pericardium can still remain moist under the premise that the amount of residual reagent meets the acceptable standard. The overall amount of residual reagent on the valve prosthesis is far below the acceptable standard. The dry valve prosthesis may be loaded or implanted directly without cleaning, which saves surgical preparation time.

(4) Clearance of valve prosthesis with stacked structure: For valve prosthesis with stacked structure, the seal element of valve prosthesis 700 is leaflet 702 and/or skirts, and the stent 701 is a multi-layer structure in the radial direction. Although the structure of each layer is open, the interlayer space is narrow, or the stacked structure made of a porous material such as fabric forms an enclosed space, in the process of valve processing, the reagent may penetrate into the narrow space or enclosed space to form residue, which is difficult to flow out of the stacked structure when cleaning, so it hard to be removed by wiping. When cleaning the gap between adjacent layers, positive pressure combined with negative pressure may be used to drive the gas phase fluid to flush and adsorb the residual reagent. For example, when cleaning a valve prosthesis with a double-layer stent and skirts made of PET fabric, a negative pressure cleaning method is used to absorb the residual reagent inside the fabric. There are a large number of reagents such as glycerin on the surface of the valve, especially in the interlayer of the double-layer stent and PET fabric. After initial wiping, the reagent in the interlayer could not be removed, and the glycerin in the PET skirts continues to seep out. The pressure is adjusted to −20 KPa, and the working head is use to absorb the reagents in the interlayer for 10 minutes. In this way, the surface of the valve prosthesis PET fabric is relatively dry, and the amount of residual reagents (such as glycerol) are far below the acceptable standard. The dry valve prosthesis may be loaded or implanted directly without cleaning, saving surgical preparation time.

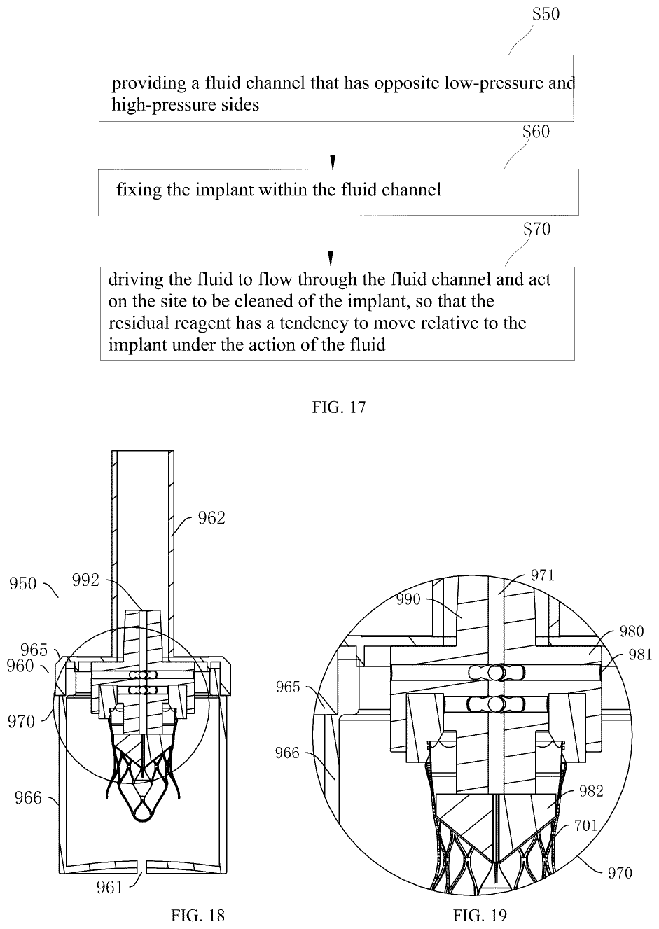

Referring to FIG. 17, an embodiment of the present disclosure provides a treatment method for an implant treated with a reagent, including:
Step S50, providing a fluid channel, wherein the fluid channel has opposite low pressure and high pressure sides;
Step S60, fixing the implant in the fluid channel; and
Step S70, driving the fluid to flow through the fluid channel and act on the site to be cleaned of the implant, so that the residual reagent has a tendency to move relative to the implant under the action of the fluid.

Specifically, the implant has opposite inflow and outflow sides, wherein the outflow side faces the high pressure side and the inflow side faces the low pressure side. Further, the method includes providing support to the implant on the inflow side, wherein a support providing support allows fluid to pass through by openings or its structure. Still further, the fluid channel is arranged along the direction of gravity, with the high pressure side facing upwardly (above the opposing gravity).

The fluid channel may be established, for example, by a deflector hood described below. Specifically, the fluid channel includes a collective space formed by the channel inside the fluid pipeline 962, the fluid channel 971, the first fluid hole, the second fluid hole, and the third fluid hole in the following embodiments.

Specifically, step S50, step S50 and step S60 each include:
Step S51, punching a hole in the fixing seat to form a fluid channel; and
Step S61, fixing a stent by a fixing seat, wherein the implant includes the stent and leaflets fixed relative to each other, and the fixing seat has a working surface (such as the end surface of the support portion 982) abutting against the leaflets and matching the shape of thereof.

Referring to FIGS. 18 and 19, the present disclosure provides an implant cleaning apparatus 950, including:
a deflector hood 960 having two opposite sides, wherein one side of which defines an opening 961 and the other side communicates with a fluid pipeline 962; and
a fixing seat 970, installed inside the deflector hood 960 and used to hold the implant. The fixing seat 970 defines a fluid channel 971 communicates with the opening 961 and the fluid pipeline 962.

The opening 961 on one side of the deflector hood 960 means that at least a portion of the side defines a hole or a hollow structure for fluid to pass through, and of course it may also be completely open.

The fluid pipeline 962 may be connected to an external negative pressure generating device. As needed, the fluid may be input into the deflector hood 960 through opening 961 by positive pressure, which is in combination negative pressure. The fluid may be air or cleaning fluid, etc.

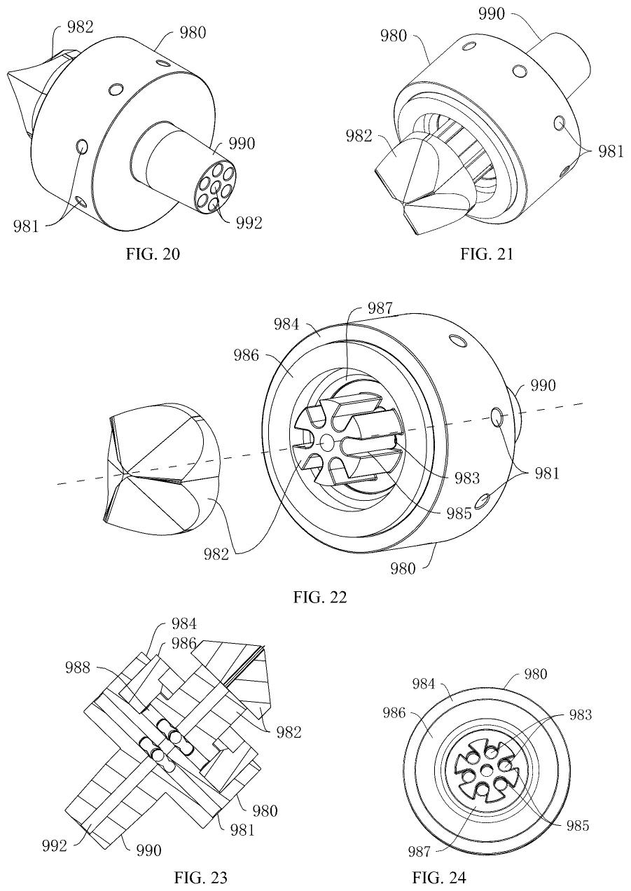

Referring to FIGS. 20 to 24, the deflector hood 960 includes a hood bottom 965 communicating with the fluid pipeline 962 and a peripheral wall 966 surrounding the hood bottom 965. The hood bottom 965 and the peripheral wall 966 are formed in one piece or separate pieces. When working, the deflector hood 960 may guide the direction of the fluid to maximize its action on the site to be cleaned of the implant.

The fixing seat 970 includes a main body 980 and a joint 990 located within the deflector hood 960. The joint 990 is connected to the main body 980 and sealingly penetrates the hood bottom 965, with the periphery of the protruding portion sealing against the bottom of the shield 965 and the protruding portion communicating with the fluid pipeline 962.

The joint and the hood bottom may be formed in one piece or separate pieces, and in case of forming in separate pieces, the two may be fixed by interference fit, adhesive fit, thread fit, or other fixing methods. Of course, the tightness therebetween may also be increased by means of an additional lock nut. Furthermore, a seal ring may be provided at the site where the fixing seat 970 contacts the hood bottom to maintain the seal.

In order to adapt to the shape of the implant, especially the shape of the site to be cleaned, the main body 980 has a support portion 982, and the end surface (working surface) of the support portion 982 abuts against the site to be cleaned of the implant.

Taking a heart valve prosthesis as an example of an implant, at least a portion of the support portion 982 is cylindrical and extends into the stent 701 of the valve prosthesis until the end surface of the support portion 982 abuts against the leaflets 702, which is the key site to be cleaned.

In order to provide positioning to the implant, the outer periphery of the support portion 982 is provided with a positioning step 984 to abut against the end surface of the implant, for example, to abut against the end surface of the stent 701. In this embodiment, the support portion 982 abuts against the inflow side 703 of the leaflet 702.

A buffer cushion 986, such as a silicone cushion, is installed on the positioning step 984. On one hand, it provides protection, and on the other hand, it can improve the positioning effect.

The outer periphery of the main body 980 may be provided with first fluid holes 981, and the end surface of the joint 990 may be provided with second fluid holes 992. Each first fluid hole 981 communicates with the second fluid hole 992 via the fluid channel 971.

The fluid channel 971 includes a main channel extending axially and branch channels radiating radially, which improve the uniformity of the fluid.

The first fluid hole 981 can provide negative pressure to drive the fluid to flow within the deflector hood 960, thereby driving the residual reagent to separate from the leaflets. In addition, the end surface of the support portion 982 may further be provided with first fluid holes 981, which allow directly suck of the leaflets to improve the cleaning effect. The first fluid holes 981 on the end surface of the support portion 982 may be configured with a smaller aperture diameter to avoid excessive local deformation of the leaflets. The support portion 982 may further be made of a porous material with supporting strength.

The second fluid hole 992 axially penetrates the main body 980, and is exposed on the side of the main body 980 facing the support portion 982 to form a third fluid hole 983. The support portion 982 has a root portion, and the outer periphery of the root portion defines grooves 985 that open radially outwardly, and each groove correspondingly communicates with the third fluid hole 983.

The third fluid hole 983 includes a central hole communicating with the main flow channel and peripheral holes arranged around the central hole. The main body 980 has an annular side wall surrounding the outer periphery of the root portion, and a radial gap 987 is formed between the annular side wall and the outer periphery of the root portion. The peripheral holes of the third fluid hole 983 are located within the radial gap 987.

Fourth fluid holes 988, which communicates with the fluid channel, may be provided on the outer periphery of the root portion. The buffer cushion 986 may avoid the fourth fluid holes 988, or be made of a porous material that allows fluid to pass through. The branch channels include two groups, with one group communicating with the fourth fluid holes and the other group communicating with the first fluid holes.

The technical features of the aforementioned embodiments may be combined arbitrarily. To simplify the description, not all the possible combinations of the technical features in the above embodiments are described. However, all of the combinations of these technical features should be considered as within the scope of the present disclosure, as long as such combinations do not contradict with each other. When the technical features in different embodiments are embodied in the same figure, it can be regarded that the figure also discloses the combination examples of the various embodiments involved.

The aforementioned embodiments only represent several embodiments of the present disclosure, and the descriptions thereof are relatively specific and detailed, but should not be construed as a limitation on the scope of the patent of the present disclosure. It should be noted that, for a person of ordinary skill in the art, several variations and improvements may be made without departing from the concept of the present disclosure, and these are all within the protection scope of the present disclosure.

What is claimed is:

1. A cleaning apparatus for treating an implant treated with a reagent, wherein the cleaning apparatus comprises:
a base;
a rotatable rack for placing one or more implants; and
a driving mechanism, coupled with the rotatable rack.

2. The cleaning apparatus of claim 1, further comprising a housing fixed to the base, wherein the rotatable rack is located inside the housing.

3. The cleaning apparatus of claim 2, wherein one side of the housing facing away from the base defines a housing opening, and the housing opening is covered with a cover body.

4. The cleaning apparatus of claim 1, wherein the driving mechanism is installed inside the base, and a control element which is electrically connected to the driving mechanism is installed on the base.

5. The cleaning apparatus of claim 3, wherein the rotatable rack comprises:
a support shaft, defining the rotation axis;
one or more holders fixed to the support shaft for loading the implant;
one or more limiting members, each cooperating with a holder and restricting the implant from detaching from the holder along a radial direction of the support shaft.

6. The cleaning apparatus of claim 5, wherein one end of the support shaft is rotatably mounted on the base, and other end of the support shaft is rotatably inserted into the cover body.

7. The cleaning apparatus of claim 3, wherein the holder is cup-shaped, comprising a cup bottom and a side wall surrounding a periphery of the cup bottom, with a cup mouth defined on a side opposite to the cup bottom, and the limiting member is engaged at the cup mouth.

8. The cleaning apparatus of claim 7, wherein the cup bottom is detachably connected to the support shaft.

9. The cleaning apparatus of claim 7, further comprising a connecting member, wherein the support shaft defines a mounting hole radially penetrating there through, the connecting member passes through the mounting hole; and wherein the holders can be arranged in pairs, with the pair of holders both connecting to the connecting member.

10. The cleaning apparatus of claim 7, wherein the cup bottom defines holes for passage of fluid, and the holes are evenly arranged in a circumferential direction at the cup bottom.

11. The cleaning apparatus of claim 7, wherein the side wall is a hollow structure, and the limiting member is a cup lid snapping fitted at the cup mouth, to prevent the implant from moving radially outwardly.

12. The cleaning apparatus of claim 11, wherein at least a portion of an outer peripheral wall of the cup lid is provided with an anti-slip member.

13. The cleaning apparatus of claim 11, wherein the cup lid is annular with an opening defined in a central portion, and one side of the cup lid opposite to the cup bottom has a positioning structure engaged with the implant.

14. The cleaning apparatus of claim 11, wherein one of the side wall and the cup lid defines a positioning groove, and other one of the side wall and the cup lid is provided with a positioning protrusion engaged with the positioning groove.

15. The cleaning apparatus of claim 14, wherein the positioning groove comprises a first groove portion extending parallel to a longitudinal axis of the holder and a second groove portion extending in a circumferential direction of the holder, and the first groove portion communicates with the second groove portion.

16. The cleaning apparatus of claim 13, wherein the positioning structure is an annular groove and/or a plurality of positioning holes, the implant comprises a stent and leaflets; and wherein the stent has connecting ears, and one of the connecting ears extends out of a corresponding positioning hole.

17. A treatment method for an implant treated with a reagent using the cleaning apparatus of claim 1, comprising:
cleaning residual reagent on a surface of the implant, wherein an adhesion force is formed between the residual reagent and the surface of the implant, the step of cleaning the residual reagent comprises: applying an external force to overcome the adhesion force,
wherein the external force is a centrifugal force, or a force generated based on fluid flow,
wherein when performing the cleaning, the implant is driven to move around a rotation axis, so that the reagent to be cleaned has a tendency to move relative to the implant, and
wherein the implant is a heart valve prosthesis, which comprises a stent and leaflets, the stent is a radially deformable tubular structure, an interior of the tubular structure defines a blood flow channel, and the leaflets are connected to the stent to control an open degree of the blood flow channel.

18. The treatment method for an implant treated with a reagent of claim 17, wherein the implant is arranged parallel or perpendicular to the rotation axis, and in case of a perpendicular arrangement, the implant has opposite inflow and outflow sides depending on control direction of blood flow by the leaflets, and wherein when the implant moves around the rotation axis, the inflow side faces the rotation axis.

19. The treatment method for an implant treated with a reagent of claim 18, wherein the implant comprises a skirt facing the rotation axis, and a side of the leaflet facing away from the rotation axis is supported by a support, which has a porous structure.

20. The treatment method for an implant treated with a reagent of claim 17, comprises rotating the implant so that the reagent to be cleaned has a tendency to move relative to the implant under a centrifugal force; wherein when rotating the implant, the rotation axis lies outside the implant, without intersecting with the implant; and wherein the implant has opposite inflow and outflow sides, and when the centrifugal force is applied, the rotation axis lies on the inflow side of the implant.
